(19) United States
(12) Patent Application Publication
(10) Pub. No.: US 2025/0154451 A1
(43) Pub. Date: May 15, 2025

(54) Biosynthesis Of Linalool

(71) Applicant: AGENCY FOR SCIENCE, TECHNOLOGY AND RESEARCH, Singapore (SG)

(72) Inventors: Congqiang Zhang, Singapore (SG); Xixian Chen, Singapore (SG); Sudha Devi D/O Manbahal Shukal, Singapore (SG); Nicola Yen Min (Chen Yuanming) Tan, Singapore (SG); Jian Ting Leonard Ong, Singapore (SG); Chin Chin Lim, Singapore (SG)

(21) Appl. No.: 18/838,822

(22) PCT Filed: Feb. 22, 2023
(86) PCT No.: PCT/SG2023/050106

§ 371 Date: Aug. 15, 2024

(30) Foreign Application Priority Data
Feb. 22, 2022 (SG) 10202201742V

(51) Int. Cl. C12N 1/20; C12N 9/10; C12N 9/88; C12N 15/52; C12P 7/04; C12R 1/19

(52) U.S. Cl. CPC C12N 1/20; C12N 9/1085; C12N 9/88; C12N 15/52; C12P 7/04; C12R 2001/19; C12Y 205/01001; C12Y 205/0101; C12Y 205/01029; C12Y 402/03025

(57) ABSTRACT

The present invention relates to host cells comprising genes of the mevalonate and linalool pathways, methods as well as kits for producing linalool.

### CROSS-REFERENCE TO RELATED APPLICATIONS

This application claims the benefit of priority of Singapore Application No. 10202201742V, filed 22 Feb. 2022, the contents of it being hereby incorporated by reference in its entirety for all purposes.

### FIELD OF THE INVENTION

The invention relates to the biosynthesis of linalool.

### BACKGROUND OF THE INVENTION

Linalool is a floral monoterpene alcohol and natural linalool can be found in many flowers (e.g., lavender and rose) or citrus fruits. Natural linalool exists in two stereoisomers, namely(S)-linalool and (R)-linalool, each bearing a unique smell and taste. Importantly, linalool is a commercially important fragrance molecule that is widely used in food, beverage, cosmetics, and personal care products (e.g., perfumes, detergents, shampoos, and lotions etc.). Linalool also serves as the starting material to the manufacturing of vitamin E.

The yield of linalool extracted from natural plant tissues is low. Thus, the current supply of linalool is predominantly produced by chemical synthesis. However, this method produces racemic linalool, which is unsustainable, environmentally unfriendly, and less favoured by consumers. Various researchers are exploring the biotechnological production of linalool, but the great majority have achieved limited success. Moreover, biosynthesis of linalool can only produce enantiopure linalool, either(S)-linalool and (R)-linalool. Therefore, there is a need to provide a novel green and cost-effective method of synthesizing linalool. that overcomes, or at least ameliorates, one or more of the disadvantages described above.

### SUMMARY

In one aspect, provided herein is a host cell comprising one or more vectors comprising a polynucleotide sequence encoding: a) mevalonate pathway genes; and b) linalool pathway genes, wherein the linalool pathway genes comprise more than one diphosphate synthase gene, prenyltransferase gene or combinations thereof, and at least one linalool synthase gene.

In another aspect, provided herein is a method of producing linalool comprising culturing the host cell as described herein in a culture medium, wherein the culture medium optionally comprises an inducer and at least one carbon substrate.

In another aspect, provided herein is a kit for producing linalool, wherein the kit comprises the host cell as described herein with instructions for use.

### Definitions

As used herein, the term “monoterpene” or “monoterpenoids” are a class of isoprenoids produced from geranyl diphosphate by various monoterpene synthases. Monoterpenoids have two isoprenoid units. Monoterpenes are secondary metabolites in plants and the main constituents of essential oils, cosmetics, food flavorings, cleaning products and drugs. They contribute to the specific smell characters of plants. Monoterpenes are industrially used as flavour, fragrant, and cosmetic constituents. Moreover, they are precursors of several flavour compounds such as citronellol, geraniol, menthol, and verbenol.

As used herein, the term “mevalonate pathway” refers to a cellular metabolic pathway that plays a key role in multiple cellular processes by synthesizing sterol isoprenoids, such as cholesterol, and non-sterol isoprenoids, such as dolichol, heme-A, isopentenyl tRNA and ubiquinone. The mevalonate pathway is the first recognized pathway for biosynthesis of isopentenyl pyrophosphate (IPP) and dimethylallyl pyrophosphate (DMAPP), which involves a series of six enzymatic steps that convert acetyl-CoA to IPP. Three molecules of acetyl-CoA are condensed to synthesize mevalonate in the first two steps of the mevalonate pathway. The enzymes acetoacetyl-CoA thiolase and HMG-COA synthase (HMGS) catalyze the condensation reactions to form hydroxymethylglutaryl-CoA (HMG-COA). Furthermore, reduction of HMG-CoA into mevalonate is catalyzed by HMG-CoA reductase (HMGR). The mevalonate thus synthesized is phosphorylated and decarboxylated to form IPP. The phosphorylation is first catalyzed by mevalonate kinase followed by the action of phosphomevalonate kinase (PMK) to form mevalonate-5-pyrophosphate. Decarboxylation is the last step, in which phosphomevalonate decarboxylase catalyzes the ATP-dependent decarboxylation of mevalonate-5-pyrophosphate to form IPP. IPP may interact with DHNA to form AQ or isomerases to form DMAPP by IPP isomerase (IDI). Genes of the mevalonate pathway refer to genes that encode enzymes of the mevalonate pathway.

As used herein, the term “linalool pathway” refers to synthesis of linalool catalyzed by linalool synthase from the precursor geranyl diphosphate (GPP). Farnesyl pyrophosphate synthase catalyzes the condensation of IPP and DMAPP units from the mevalonate pathway to generate prenyl diphosphate intermediates of different chain length. Condensation of IPP and DMAPP yields geranyl diphosphate (GPP) as the precursor to monoterpenoids. Linalool synthase then catalyzes the conversion of geranyl diphosphate to linalool. Genes of the linalool pathway refer to genes that encode enzymes of the linalool pathway.

As used herein, the term “polypeptides” includes polypeptides, proteins, peptides, fragments of polypeptides, and fusion polypeptides.

As used herein, a “nucleic acid” refers to two or more deoxyribonucleotides and/or ribonucleotides covalently joined together in either single or double-stranded form.

As used herein, the term “operably linked” refers to a functional linkage between a nucleic acid expression control sequence (such as a promoter) and a second nucleic acid sequence, wherein the expression control sequence regulates the transcription of the nucleic acid corresponding to the second sequence.

As used herein, the term “variant” refers to a modification in the DNA sequence. The modification in the DNA sequence includes mutation, truncation, translocation, substitution, deletion, and insertion, resulting in the alteration of the activity of the gene.

The term “promoter” as used herein refers to a region of the DNA that initiates transcription of a gene. The region of the DNA is typically located near the transcription start site of a gene and upstream on the DNA. A promoter may be inducible or non-inducible. The term “inducible promoter” as used herein refers to a promoter that can be regulated in the response to specific stimuli, also known as inducers. The promoter system may be modified to be inducible. Examples of inducible promoter systems include the Tet-on system, Tet-off system, T7 system, Trp system, Tac system, lambda cl857-PL system, bacterial EL222 system and Lac system. A promoter may also be a constitutive promoter which is a promoter that is always active.

As used herein, the term “promoter engineering” refers to the use of different promoters for each operon or a single gene in the plasmid to optimize the levels of gene expression.

The term “ribosomal binding site” as used herein in the context of the application refers to a site of an mRNA molecule which recruits and binds the ribosome, allowing the selection of the proper initiation codon during the initiation of translation. The ribosomal binding site controls the accuracy and efficiency of the initiation of mRNA translation.

As used herein, the term “titration by ribosomal binding site (RBS) engineering” refers to alteration of a RBS using a synthetic RBS library consisting of RBSs with varying translation rates to modulate the expression of a gene in the biosynthetic pathway.

As used herein, the term “linker” refers to short amino acid sequences that separate multiple domains in a recombinant or fusion protein. Linkers function to prohibit unwanted interactions between the discrete domains. However, there are flexible Gly-rich linkers that connect various domains in a single protein without interfering with the function of each domain. Gly-rich linkers can also help create a covalent link between proteins to form a stable protein-protein complex. The lengths of linkers vary from 2 to 31 amino acids, optimized for each condition so that the linker does not impose any constraints on the conformation or interactions of the linked partners.

As used herein, the term “deficient” in the context of the expression of a gene or protein refers to a reduction in expression level of a gene or protein relative to a baseline level of expression of the gene or protein. Deficient in the context of the expression of a gene or protein may also refer to non-expression of a gene or protein in a scenario where the gene or protein would otherwise be expressed. The baseline expression of a gene or a protein would be understood to mean the expression level of an unmutated gene or a wild type gene, or in the context where the gene or protein would otherwise be expressed, the expression level of the gene or protein.

As used herein, the term “about”, is used in the context of, but not limited to, concentrations of components and percentages of compounds, typically refers to +/−10% of the stated value, to +/−9% of the stated value, to +/−8% of the stated value, to +/−7% of the stated value, to +/−6% of the stated value, to +/−5% of the stated value, +/−4% of the stated value, more typically +/−3% of the stated value, more typically, +/−2% of the stated value, even more typically +/−1% of the stated value, and even more typically +/−0.5% of the stated value. Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1 depicts the linalool production (titres, OD₆₀₀ and specific yield) by the optimizing metabolic pathways. Nine constructs were tested by varying promoter strength for the mevalonate pathways.

FIG. 2 shows the linalool production and biomass by optimization of induction. FIG. 2A shows the time of induction. FIG. 2B shows the optimization of IPTG concentration. FIG. 2C shows the optimization of lactose concentration.

FIG. 3 illustrates engineering GPP synthase (GPPS) and linalool synthase to boost linalool yields. FIG. 3A shows the engineering approaches (protein fusion, truncation, and pathway redesign). FIG. 3B depicts the linalool production of different constructs using truncated ApLS and fusion of ispA and ApLS. FIG. 3C depicts the supplement of an additional GPPS AgGPPS.

FIG. 4 depicts the comparison of various media and enhancing linalool titre by optimization of carbon substrates. FIG. 4A shows the linalool production in three media: 2xPY, TB and defined media. FIG. 4B shows the optimization of the concentration of carbon sources.

FIG. 5 refers to a summary of all the strategies. Yields are defined as the ratio of the linalool produced and the total carbons supplied in the medium.

FIG. 6 depicts an example of the sps004 plasmid map used.

FIG. 7 depicts an example of the ApLS-ispA_S80F-AgGPPS plasmid map used.

### DETAILED DESCRIPTION OF THE PRESENT INVENTION

In a first aspect the present invention refers to a host cell comprising one or more vectors comprising a polynucleotide sequence encoding: a) mevalonate pathway genes; and b) linalool pathway genes, wherein the linalool pathway genes comprise more than one diphosphate synthase gene, prenyltransferase gene or combinations thereof, and at least one linalool synthase gene.

The mevalonate pathway genes and the linalool pathway genes may be encoded on one or more vectors within the host cell. For example, the mevalonate pathway genes and linalool pathway genes may be encoded on one vector, two vectors, three vectors, four vectors, five vectors or six vectors. It will be appreciated by a person skilled in the art that the mevalonate pathway genes and the linalool pathway genes can be located in one or more vectors in different combinations. In one example, the mevalonate pathway genes may be encoded on one vector and the linalool pathway genes may be encoded on another vector. In another example, the mevalonate pathway genes may be encoded on two vectors and the one or more genes of the linalool pathway genes may be encoded on another vector. In another example, the linalool pathway genes may be encoded on two vectors and the mevalonate pathway genes may be encoded on another vector. It will also be appreciated by a person skilled in the art that where there is more than one gene of a pathway, these can be encoded on separate vectors in combination with one or more genes from another pathway. It will generally be understood that the examples provided in the foregoing are not exhaustive and different combinations would be acceptable.

The mevalonate pathway genes may be encoded by one or more polynucleotide sequences on one or more vectors, and the linalool pathway genes may be encoded by one or more polynucleotide sequences on one or more vectors. The one or more polynucleotide sequences may encode two, three, four, five, six, seven, eight, nine, ten or more mevalonate pathway genes, and two, three, four, five, six, seven, eight, nine, ten or more linalool pathway genes on one or more vectors. It will be appreciated by a person skilled in the art that the one or more polynucleotide sequences may encode mevalonate pathway genes and the linalool pathway genes in different combinations on one or more vectors. In one example, the host cell may comprise one vector, wherein the one or more polynucleotide sequences on the vector may encode two mevalonate pathway genes and two linalool pathway genes. In another example, the host cell may comprise two vectors, wherein the one or more polynucleotide sequences on the first vector may encode two mevalonate pathway genes and the one or more polynucleotide sequence on the second vector may encode two linalool pathway genes. In another example, the host cell may comprise one vector, wherein the one of more polynucleotide sequences on the vector may encode three mevalonate pathway genes and three linalool pathway genes. In another example, the host cell may comprise two vectors, wherein the one or more polynucleotide sequences on the first vector may encode two mevalonate pathway genes and the one or more polynucleotide sequences on the second vector may encode three linalool pathway genes. In another example, host cell may comprise two vectors, wherein the one or more polynucleotide sequences may encode two mevalonate pathway genes and two linalool pathway genes on the first vector and the one or more polynucleotide sequences on the second vector may encode one mevalonate pathway gene and one linalool pathway gene. In another example, the host cell may comprise three vectors, wherein one or more polynucleotide sequences on the first vector may encode two mevalonate pathway genes, one or more polynucleotide sequences on the second vector may encode two linalool pathway genes and one or more polynucleotide sequences on the third vector may encode three mevalonate pathway genes. It will generally be understood that the examples provided in the foregoing are not exhaustive and different combinations would be acceptable. The two, three, four, five, six, seven, eight, nine, ten or more linalool pathways genes and two, three, four, five, six, seven, eight, nine, ten or more mevalonate genes may in some examples be inserted into the genome of the host cell. A person skilled in the art would understand that the genomic insertion of linalool pathway genes and mevalonate pathway genes into the host genome refers to the targeted and stable insertion of an exogenous gene into the host genome, allowing stable gene expression. Linalool pathway genes and mevalonate pathway genes may be inserted into the genome of the host cell using genomic modification methods including but is not limited to CRISPR-Cas9, TALEN-mediated gene knockin.

It would generally be understood that the two, three, four, five, six, seven, eight, nine, ten or more linalool pathway genes and the two, three, four, five, six, seven, eight, nine, ten or more mevalonate pathway genes may be encoded by one or more polynucleotide sequences on one or more vectors or may be inserted into the genome of the host cell or may be encoded by one or more polynucleotide sequences on one or more vectors and inserted into the genome of the host cell.

In one example, the host cell may comprise one or more vectors wherein a) the mevalonate pathway genes are encoded by one or more polynucleotide sequences on one or more vectors; and b) the linalool pathway genes are encoded by one or more polynucleotide sequences on one or more vectors.

In one example, the host cell may comprise mevalonate pathway genes and linalool pathway genes that are inserted into the genome of the host cell.

In one example, the host cell may comprise one or more vectors wherein a) the mevalonate pathway genes are encoded by one or more polynucleotide sequences on one or more vectors; and b) the linalool pathway genes are inserted into the genome of the host cell.

In some examples, the mevalonate pathway genes are isolated from bacterium or yeast. In one example, the mevalonate pathway genes may be isolated from a bacterium selected from the group consisting of Escherichia coli, Pantoea agglomerans, Pantoea ananatis, uncultured marine bacterium HF10_19P19, Sulfolobus solfataricus, Anabaena variabilis and Brevundimonas sp. In one example, the mevalonate pathway genes may be isolated a yeast selected from the group consisting of Saccharomyces cerevisiae Yarrowia lipolytica, Rhodosporidium toruloides, Candida and Pichia.

Genes of the mevalonate pathway include but are not limited to HMG-COA synthase (hmgS), acetoacetyl-CoA thiolase (atoB), HMG-COA reductase (hmgR), mevalonate kinase (mevk), phosphomevalonate kinase (pmK), mevalonate pyrophosphate decarboxylase (pmd), (isopentenyl diphosphate) IPP isomerase (idi), isopentenyl phosphate kinase, mevalonate 3-phosphate kinase, choline kinase, and acid phosphatase.

In one example, the mevalonate pathway genes may be selected from the group consisting of HMG-COA synthase (hmgS), acetoacetyl-CoA thiolase (atoB), HMG-COA reductase (hmgR), mevalonate kinase (mevK), phosphomevalonate kinase (pmK), mevalonate pyrophosphate decarboxylase (pmd), (isopentenyl diphosphate) IPP isomerase (idi) or combinations thereof.

It will be appreciated by a person skilled in the art that the mevalonate pathway genes may encoded by one or more polynucleotide sequences on one or more vectors in various combinations. For example, the one or more polynucleotide sequences encoding atoB, hmgS, hmgR, mevk, pmK, pmd and idi are located on one vector. In another example, the polynucleotide sequence encoding atoB is on one vector, and the one or more polynucleotide sequences encoding the hmgS, hmgR, mevk, pmK, pmd and idi are located on a second vector. In another example, the one or more polynucleotide sequences encoding atoB and hmgS are located on one vector and the one or more polynucleotide sequences encoding hmgR, mevk, pmK, pmd and idi are located on a second vector. It will generally be understood that the examples provided in the foregoing are not exhaustive and different combinations would be acceptable.

In one example, the polynucleotide sequence encoding the atoB, hmgS, hmgR, mevk, pmK, pmd and idi genes of the mevalonate pathway is located on one vector.

In one example, the polynucleotide sequence encoding the atoB, hmgS, hmgR genes of the mevalonate pathway is on a first vector, and the polynucleotide sequence encoding the mevk, pmK, pmd and idi genes of the mevalonate pathway is on a second vector.

In one example, the polynucleotide sequence encoding atoB gene is SEQ ID NO: 1. In one example, the polynucleotide sequence encoding hmgS gene is SEQ ID NO: 2. In one example, the polynucleotide sequence encoding mevK gene is SEQ ID NO: 3. In one example, the polynucleotide sequence encoding pmK gene is SEQ ID NO: 4. In one example, the polynucleotide sequence encoding pmd gene is SEQ ID NO: 5. In one example, the polynucleotide sequence encoding idi gene is SEQ ID NO: 6.

The mevalonate pathway genes may in some examples be modified. The modification of the one or more genes may comprise mutation, truncation, translocation, substitution, deletion and insertion, or post-translation modification of the translated gene. The genes may be modified to improve the expression levels. post-translational modification of the translated protein or combinations of any of these modifications.

In one example, the hmgR gene is truncated. It will be appreciated by a person skilled in the art that the term ‘truncation’ refers to elimination of the N- or C-terminal portion of a protein by manipulation of the structural gene, or premature termination of protein elongation due to the presence of a termination codon in its structural gene as a result of a nonsense mutation. In one example, the polynucleotide sequence encoding truncated hmgR is SEQ ID NO: 7 and the polypeptide sequence of truncated hmgR is SEQ ID NO: 8.

In some examples, mevalonate pathway genes may be wild-type genes only, mutated genes only or combinations thereof. For example, the host cell may express wild-type atoB, hmgS, hmgR, mevK, pmK, pmd and idi. In another example, the host cell may express mutated atoB, hmgS, hmgR, mevK, pmK, pmd and idi. In yet another example, the host cell may express wild-type atoB, hmgS, mevk, pmK, pmd, idi, and mutated hmgR. It will generally be understood that the examples provided in the foregoing are not exhaustive and different combinations would be acceptable. In a preferred example, the host cell expresses wild-type atoB, hmgS, mevK, pmK, pmd, idi, and truncated hmgR.

In some examples, the linalool pathway genes are isolated from a eukaryote or a prokaryote, or combinations thereof. The eukaryote may be a fungus or a plant. The prokaryote may be a bacterium. In some examples, the linalool pathway genes may be isolated from a plant including but not limited to Lavandula angustifolia, Mentha aquatica, Cinnamomum camphora, Zea mays, Coffea arabica, Wurfbainia villosa, Zizania palustris, Zingiber officinale, Vitis vinifera, Vigna unguiculata, Triticum urartu, Achillea millefolium subsp. millefolium, Actinidia arguta, Actinidia polygama, Aegilops tauschii, Albizia julibrissin, Amborella trichopoda, Ananas comosus var. bracteatus, Aquilegia coerulea, Artemisia annua, Dendrobium catenatum, Trifolium subterraneum, Trema orientale Triticum aestivum, Tetracentron sinense, Tanacetum cinerariifolium, Backhousia citriodora, Brachypodium distachyon, Brassica campestris, Cajanus cajan, Camellia saluenensis, Camellia sinensis, Cannabis sativa, Capsella rubella, Cinnamomum camphora, Cinnamomum micranthum f. kanehirae, Cinnamomum osmophloeum, Citrus unshiu, Clarkia concinna, Corchorus olitorius, Coriandrum sativum, Striga asiatica, Spirodela intermedia, Salvia dorisiana, Salix suchowensis, Rosa rugosa, Rosa chinensis, Rhododendron simsii, Rhodamnia argentea, Raphanus sativus, Pyrus ussuriensis x Pyrus communis, Dichanthelium oligosanthes, Digitaria exilis, Prunus yedoensis var. nudiflora, Prunus persica, Prunus dulcis, Ficus carica, Freesia hybrid cultivar, Glycine soja, Handroanthus impetiginosus, Hedychium coronarium, Hevea brasiliensis, Lavandula angustifolia, Lavandula latifolia, Lavandula x intermedia, Leersia perrieri, Magnolia champaca, Malus domestica, Populus deltoides, Picea sitchensis, Picea glauca, Picea abies, Microthlaspi erraticum, Mikania micrantha, Miscanthus lutarioriparius, Morella rubra, Morus notabilis, Mucuna pruriens, Ocimum basilicum, Opuntia streptacantha, Phyla dulcis, Perilla frutescens var. hirtella, Parasponia andersonii, Panicum virgatum, Osmanthus fragrans var. thunbergii, Oryza sativa subsp. Japonica, Oryza punctata, Oryza meyeriana var. granulate, and Oryza brachyantha. In other examples, the linalool pathway genes may be isolated from a fungus including but not limited to Agrocybe pediades, Galerina marginate, Hypholoma sublateritium, Hebeloma cylindrosporum, Lactarius deliciosus, Psilocybe cyanescens, Hypholoma fasciculare, Dictyostelium discoideum, Aspergillus calidoustus, Puccinia graminis f. sp. tritici, Melampsora larici-populina, Physarum polycephalum, Paxillus rubicundulus, and Postia placenta. In yet other examples, the linalool pathway genes may be isolated from a bacterium including but not limited to Escherichia coli, Methanothrix sp, Oceanospirillales bacterium, Oleispira sp, and Streptomyces clavuligerus.

Genes of the linalool pathway include but are not limited to linalool synthase (LIS or LS), IspA, g9127, Psicya1, Psicya2 and QDF59315. Linalool pathway genes may also include diphosphate synthase and prenyltransferase. The more than one diphosphate synthase gene, prenyltransferase gene or combination of diphosphate synthase and prenyltransferase genes of the linalool pathway in the host cell include but are not limited to a geranyl pyrophosphate synthase (GPPS), farnesyl diphosphate synthase (FPPS) or combinations thereof.

The linalool pathway genes may in some examples be modified. Modification may comprise mutation, truncation, translocation, substitution, deletion and insertion, or post-translation modification of the translated gene. The genes may be modified to improve the expression levels, post-translational modification of the translated protein or combinations of any of these modifications.

In one example, the linalool synthase gene is isolated from Agrocybe pediades (ApLS). In other examples, the linalool synthase gene is truncated. It will be appreciated by a person skilled in the art that the term ‘truncation’ refers to elimination of the N- or C-terminal portion of a protein by manipulation of the structural gene, premature termination of protein elongation due to the presence of a termination codon in its structural gene as a result of a nonsense mutation or proteolysis of the protein. In one example, ApLS is truncated at the C-terminal end. In another example, the first 10 or 19 amino acids from the C-terminal end of ApLS is truncated and the truncated ApLS comprises the polynucleotide sequence as set forth in SEQ ID NO: 9 or SEQ ID NO: 10. In another example, the truncated ApLS comprises the polypeptide sequence as set forth in SEQ ID NO: 11.

In some examples, the more than one GPPS may be isolated from a prokaryote or a eukaryote or a combination of a prokaryote and eukaryote. The eukaryote may be a fungus or a plant. The prokaryote may be a bacterium. In some examples, the more than one GPPS may be isolated from a fungus including but not limited to Neocamarosporium betae, Phomopsis amygdali and Zymoseptoria tritici. In other examples, the more than one GPPS may be isolated from a plant including but not limited to Mentha piperita, Arabidopsis thaliana, Abies grandis, Antirrhimum majus, and Clarkia breweri. In yet other examples, the more than one GPPS may be isolated from a bacterium including but not limited to Escherichia coli, Streptomyces mobaraensis and Streptomyces niveus. It will be appreciated by a person skilled in the art that the more than one GPPS may be isolated from the same source or a combination of different sources. The more than one GPPS may be isolated from a single prokaryote or different prokaryotes. For example, the more than one GPPS may be isolated from Escherichia coli only. In another example, the more than one GPPS may be isolated from Escherichia coli and Streptomyces mobaraensis. The more than one GPPS may be isolated from a single eukaryote or different eukaryotes. For example, the more than one GPPS may be isolated from Abies grandis only. In another example, the more than one GPPS may be isolated from Abies grandis and Arabidopsis thaliana. The more than one GPPS may also be isolated from a combination of prokaryote and eukaryote. For example, the more than one GPPS may be isolated from Escherichia coli and Abies grandis. It will generally be understood that the examples provided in the foregoing are not exhaustive and different combinations would be acceptable. In a preferred example, the GPPS is isolated from Escherichia coli or Abies grandis (AgGPPS) or a combination of Escherichia coli and Abies grandis.

In some examples, the more than one GPPS gene is isolated from Escherichia coli and may be a farnesyl pyrophosphate synthase. In one example, the farnesyl pyrophosphate synthase may be but is not limited to IspA.

The GPPS gene isolated from Escherichia coli may be a farnesyl pyrophosphate synthase mutated at one or more amino acid position. In one example, the mutated GPPS is IspAS80F.

In one example, the IspAS8OF comprises the polypeptide sequence as set forth in SEQ ID NO. 12.

In some examples, the more than one GPPS gene is isolated from Abies grandis (AgGPPS). The AgGPPS may be truncated at the N-terminal end.

In some examples, the AgGPPS is truncated between amino acid positions 2 to 86. In one example, the AgGPPS may be truncated from amino acid positions 2 to 10. In another example, the AgGPPS may be truncated from amino acid positions 2 to 20. In another example, the AgGPPS may be truncated from amino acid positions 2 to 50. In another example, the AgGPPS may be truncated from amino acid positions 2 to 70. It will generally be understood that the examples provided in the foregoing are not exhaustive and different combinations would be acceptable. In a preferred example, the AgGPPS is truncated from amino acid positions 2 to 86.

In one example, the truncated AgGPPS comprises the polypeptide sequence as set forth in SEQ ID NO: 13.

In some examples, linalool pathway genes may be wild-type genes only, mutated genes only or combinations thereof. For example, the host cell may express wild-type ApLS, ispA and AgGPPS. In another example, the host cell may express mutated ApLS, ispA and AgGPPS. In yet another example, the host cell may express wild-type ApLS, mutated ispA and AgGPPS. It will generally be understood that the examples provided in the foregoing are not exhaustive and different combinations would be acceptable. In a preferred example, the host cell expresses wild-type ApLS, mutated ispA and truncated AgGPPS.

In some examples, the polynucleotide sequence encoding the ApLS gene, the IspAS8OF gene, and the truncated AgGPPS gene may be located on one or more vectors, inserted into the genome of the host cell or combinations thereof. In one example, the polynucleotide sequence encoding the ApLS gene and the IspAS8OF gene may be located on one or more vectors, and the truncated AgGPPS gene of the linalool pathway is inserted into the genome of the host cell. In another example, the polynucleotide sequence encoding the ApLS gene, IspAS80F and the truncated AgGPPS gene of the linalool pathway are inserted into the genome of the host cell. In a preferred example, the polynucleotide sequence encoding the ApLS gene, the IspAS8OF gene, and the truncated AgGPPS gene of the linalool pathway is on one vector. It will generally be understood that the examples provided in the foregoing are not exhaustive and different combinations would be acceptable.

In one example, the host cell comprises
a) a first vector comprising a polynucleotide sequence encoding atoB, hmgS and truncated hmgR genes of the mevalonate pathway;
b) a second vector comprising a polynucleotide sequence encoding mevk, pmK, pmd and idi genes of the mevalonate pathway;
c) a third vector comprising a polynucleotide sequence encoding ApLS gene, IspAS8OF gene, and truncated AgGPPS gene of the linalool pathway.

The polynucleotides sequences in each of the one or more vectors would be understood to be operably linked to a promoter. It would generally be understood that any promoter that allows expression of the polynucleotide sequence may be employed. Examples of promoters include but are not limited to the T7 RNA polymerase promoter, the lac promoter, araBAD promoter, tac promoter, lambda cl857-PL promoter and the T5 promoter.

In some examples, the promoter may be an inducible promoter. In one example, the promoter may be naturally inducible. In one example, the promoter may be engineered to be inducible. It will be appreciated that any suitable inducible promoter system may be used. Inducible promoter systems may be induced by an inducer or stimuli including but not limited to chemical inducers, light or heat.

In one example, the polynucleotide sequence is operably linked to an inducible promoter in one or more vectors and operably linked to an uninducible promoter in the other vectors. For example, the polynucleotide sequence is operably linked to an inducible promoter in each of the vectors. In another example, the polynucleotide sequence is operably linked to an inducible promoter in two vectors and the polynucleotide sequence is operably linked to an uninducible promoter in the other vectors.

In one example, the polynucleotide sequence in each of the vectors is operably linked to an inducible promoter. In one example, the inducible promoter is a wild-type T7 RNA polymerase promoter or a variant of the wild-type T7 RNA polymerase promoter. The variant of the wild-type T7 RNA polymerase promoter may be generated via mutations to the wild-type promoter. In another example, the T7 RNA polymerase promoter variant is selected from the group consisting of TM1, TM2, TM3, TV1, TV2, TV3 and TV4. In one example, the polynucleotide sequence encoding wild-type T7 RNA polymerase promoter is SEQ ID NO: 14. In one example, the polynucleotide encoding the TM1 promoter is SEQ ID NO: 15. In one example, the polynucleotide encoding the TM2 promoter is SEQ ID NO: 16. In one example, the polynucleotide encoding the TM3 promoter is SEQ ID NO: 17. In one example, the polynucleotide sequence encoding the TV1 promoter is SEQ ID NO: 18. In one example, the polynucleotide sequence encoding the TV2 promoter is SEQ ID NO: 19. In one example, the polynucleotide sequence encoding the TV3 promoter is SEQ ID NO: 20. In one example, the polynucleotide sequence encoding the TV4 promoter is SEQ ID NO: 21.

The inducible promoter in each of the vectors may be independently selected from the wild-type T7 RNA polymerase promoter or variants. The variant of the wild-type T7 RNA polymerase promoter may comprise a polynucleotide sequence with at least one mutation. The polynucleotide sequence of the variant T7 RNA polymerase promoter may comprise one, two, three, four, five, six, or more mutations. In some examples, the polynucleotide sequence of the variant T7 RNA polymerase promoter may be taatacgactcX₁X₂X₃X₄X₅X₆ggggaattgtgagc as set forth in SEQ ID NO. 22, wherein ‘X₁-X₆’ can represent any nucleotide. It will generally be understood that different combinations of the mutated polynucleotide sequence of T7 RNA polymerase promoter would be acceptable.

In one example, the inducible promoter in each of the vectors may be the wild-type T7 RNA polymerase promoter. In another example, the inducible promoter in each of the vectors may be the same T7 RNA polymerase promoter variant. In yet another example, the inducible promoter in each of the vectors may be different or combinations of the wild-type T7 RNA polymerase promoter and variants. It will generally be understood that apart from the examples provided herein, different combinations of inducible promoters may be used with each of the vectors of the invention.

In some examples, the one or more inducible promoter operably linked to the polynucleotide sequence encoding the mevalonate pathway genes, the polynucleotide sequence encoding the linalool pathway genes, or the polynucleotide sequence encoding the mevalonate pathway genes and the polynucleotide sequence encoding the linalool pathway genes is engineered to balance the mevalonate pathway, the linalool pathway or the mevalonate pathway and linalool pathway.

In one example, the first vector may comprise a) a polynucleotide sequence encoding the hmgS, atoB, hmgR genes of the mevalonate pathway operably linked to a first inducible promoter; and b) a polynucleotide sequence encoding the mevk, pmK, pmd and idi genes of the mevalonate pathway operably linked to a second inducible promoter.

In one example, the inducible promoter in the first vector comprising the polynucleotide sequence encoding atoB, hmgS and truncated hmgR genes of the mevalonate pathway in the host cell as described herein is TM1 and the inducible promoter in the first vector comprising the polynucleotide sequence encoding mevk, pmK, pmd and idi genes of the mevalonate pathway in the host cell as described herein is TM1. In one example, the inducible promoter in the first vector comprising the polynucleotide sequence encoding atoB, hmgS and truncated hmgR genes of the mevalonate pathway in the host cell as described herein is TM1 and the inducible promoter in the first vector comprising the polynucleotide sequence encoding mevk, pmK, pmd and idi genes of the mevalonate pathway in the host cell as described herein is TM2. In one example, the inducible promoter in the first vector comprising the polynucleotide sequence encoding atoB, hmgS and truncated hmgR genes of the mevalonate pathway in the host cell as described herein is TM3 and the inducible promoter in the first vector comprising the polynucleotide sequence encoding mevk, pmK, pmd and idi genes of the mevalonate pathway in the host cell as described herein is TM2. In one preferred example, the inducible promoter in the first vector comprising the polynucleotide sequence encoding atoB, hmgS and truncated hmgR genes of the mevalonate pathway in the host cell as described herein is TM2 and the inducible promoter in the first vector comprising the polynucleotide sequence encoding mevk, pmK, pmd and idi genes of the mevalonate pathway in the host cell as described herein is TM1. It will generally be understood that apart from the examples provided herein, different combinations of inducible promoters may be used with each of the vectors of the invention.

In some examples, the genes of the host cell are titrated by RBS engineering. The one or more vectors in the host cell as described herein may further comprise a polynucleotide sequence encoding a ribosomal binding site (RBS). Each vector in the host cell may further comprise the polynucleotide sequence encoding the RBS or some of the vectors may further comprise the polynucleotide sequence encoding the RBS while the others do not. For example, each of the first and second vectors may further comprise the polynucleotide sequence encoding the RBS. In another example, the first vector may comprise the polynucleotide sequence encoding the RBS while the second vector does not. In a preferred example, the polynucleotide sequence encoding the RBS is located on the vector encoding the linalool pathway genes, wherein the linalool pathway genes comprise more than one GPPS.

It will be appreciated by one of skill in the art that the sequence encoding the RBS may be optimized for translational efficiency and the strength of the RBS with respect to the polynucleotide sequence to be translated. Optimization of a RBS would generally be understood to involve modification of the polynucleotide sequence of the RBS. The RBS may be modified by substitution, deletion, insertion, or combinations thereof of one or more nucleotide bases. The RBS may be modified using degenerate oligonucleotide bases.

The polynucleotide sequence encoding the RBS may be synthesized and inserted upstream of the genes located in one or more vectors. For example, the RBS may be synthesized and inserted upstream of the genes in two vectors. In another example, the polynucleotide sequence encoding the RBS may be synthesized and inserted upstream the genes in one vector. It will generally be understood that the examples provided in the foregoing are not exhaustive and different combinations would be acceptable.

In some examples, the RBS may comprise a polynucleotide sequence of SEQ ID NO: 23, SEQ ID NO: 24, SEQ ID NO: 25 or SEQ ID NO: 26. In a preferred example, the RBS comprises a polynucleotide sequence of SEQ ID NO: 23 or SEQ ID NO: 24.

In one example, the polynucleotide sequence encoding the RBS of the GPPS isolated from Escherichia coli comprises the SEQ ID NO: 23 and the polynucleotide sequence encoding the RBS of AgGPPS comprises the SEQ ID NO: 24.

In some examples, the host cell may be deficient in at least one gene involved in amino acid degradation. The host cell may be deficient in one, two, three, four, five, six, seven, eight, nine, ten or more genes involved in amino acid degradation. It will generally be understood that the examples provided in the foregoing are not exhaustive.

The gene involved in amino acid degradation may be but is not limited to tnaA.

The host cell may be modified using genomic modification methods to be deficient in at least one gene involved in amino acid degradation. Reduction in gene expression levels may be carried out using genomic modification methods including but is not limited to siRNA knockdown and shRNA knockdown. The at least one gene may be deleted from the genome of the host cell using genomic modification methods including but is not limited to CRISPR-Cas9, FRT gene deletion, TALEN-mediated gene knockout

As described herein, the host cell of the present invention may be a bacterial cell. The bacterial cell may be but is not limited to Escherichia, Pantoea, Bacillus, Corynebacterium, Paracoccus, Streptomyces and Synechococcus. In a preferred example, the bacterial cell is an Escherichia coli cell. It will generally be understood that any industrial bacterium or bacterial cell may be used in the present invention.

The strain of the Escherichia coli cell may be but is not limited to BL21 DE3 strain, K-12 (RV308), K-12 (HMS174), K-12 substr. MG1655, W strain (ATCC 9637), JM109 (DE3), BW25113, JM109 DE3, Mach1, DH10B, JM109 and any industrial strain. In a preferred example, the Escherichia coli cell is a BL21 DE3 strain.

In another aspect, the present invention refers to a method of producing linalool comprising culturing the host cell as described herein in a culture medium. The host cell may be cultured in a suitable culture vessel including but not limited to a tube, a flask or a bioreactor.

The method of linalool production may further comprise the step of isolating linalool from the culture medium.

The method comprises the culturing of the host cell as described herein in a culture medium. The culture medium may comprise but not limited to components in the TB medium and the 2XPY medium. Additional components may be added to the culture medium and include antibiotics, inducers, and carbon substrates.

The antibiotics may be supplemented in the culture medium at the beginning of the culturing process. The antibiotics may be added continuously throughout the culturing process. Examples of antibiotics that may be used include but are not limited to kanamycin, spectinomycin, ampicillin, bleocin, carbenicillin, chloramphenicol, coumermycin, gentamycin, and tetracycline. In a preferred example, the antibiotic is kanamycin or spectinomycin.

The culture medium may be further supplemented by one or more inducers capable of inducing the inducible promoter. The inducer may be added in the culture medium at the beginning of the of the culturing process. The culture medium may be supplemented with the inducer when the host cell has grown to an optical density. The culture medium may be supplemented continuously to the culture medium throughout the culturing process. In another example, the host cell may be cultured in conditions suitable for inducing the inducible promoter.

Examples of inducers include but are not limited to galactose, lactose or isopropyl β-D-1 thiogalactopyranoside (IPTG). In a preferred example, the inducer is lactose or IPTG.

The concentration of IPTG may be about 0.01 mM to about 0.3 mM. For example, the concentration of IPTG may be about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, about 0.11 mM, about 0.12 mM, about 0.13 mM, about 0.14 mM, about 0.15 mM, about 0.16 mM, about 0.17 mM, about 0.18 mM, about 0.19 mM, about 0.20 mM, about 0.21 mM, about 0.22 mM, about 0.23 mM, about 0.24 mM, about 0.25 mM, about 0.26 mM, about 0.27 mM, about 0.28 mM, about 0.29 mM, about 0.30 mM. In a preferred example, the concentration of IPTG is about 0.20 mM.

The culture medium in a tube or in a flask may be supplemented with an inducer when the host cell has grown to an optical density (OD₆₀₀) of about 1 to about 5. The optical density may be about 1.0, about 1.5, about 2.0, about 2.5, about 3.0, about 3.5, about 4.0, about 4.5 and about 5.0. In a preferred example, the OD₆₀₀ is about 2.0.

The fed batch fermentation culture medium in the bioreactor may be supplemented with an inducer when the host cell has grown to an optical density (OD₆₀₀) of about 20 to about 60. The optical density may be about 20, about 25, about 30, about 35, about 40, about 45, about 50, about 55 and about 60.

The culture medium may also comprise at least one carbon substrate which may be but is not limited to glucose, glycerol, lactose, and sucrose. A person skilled in the art will understand that the culture medium may contain a single type of carbon substrate or combinations of carbon substrates. In a preferred example, the culture medium comprises lactose, glucose and glycerol.

The concentration of the carbon substrate may be about 1 to about 50 g/L. For example, the concentration of the carbon substrate may be about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, about 45 g/L, about 46 g/L, about 47 g/L, about 48 g/L, about 49 g/L and about 50 g/L.

The concentration of lactose may be about 5 mM to about 80 mM. For example, the concentration of lactose may be about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM and about 80 mM. In a preferred example, the concentration of lactose is about 40 mM.

The concentration of glucose may be about 1 g/L to about 50 g/L. For example, the concentration of glucose may be about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, about 45 g/L, about 46 g/L, about 47 g/L, about 48 g/L, about 49 g/L and about 50 g/L. In a preferred example, the concentration of glucose is about 3 g/L.

The concentration of glycerol may be about 1 g/L to about 50 g/L. For example, the concentration of glycerol may be about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 41 g/L, about 42 g/L, about 43 g/L, about 44 g/L, about 45 g/L, about 46 g/L, about 47 g/L, about 48 g/L, about 49 g/L and about 50 g/L. In a preferred example, the concentration of glycerol is about 12 g/L.

The method of linalool production in some examples, comprise culturing the host cell in the culture medium for about 1 day, about 2 days, about 3 days, about 4 days, about 5 days, about 6 days and about 7 days. It will generally be understood that the examples provided in the foregoing are not exhaustive.

The culture medium may be maintained at a pH of about 6.5 to about 7.8. For example, the pH may be about 6.5, about 6.6, about 6.7, about 6.8, about 6.9, about 7.0, about 7.1, about 7.2, about 7.3, about 7.4, about 7.5, about 7.6, about 7.7 and about 7.8. In a preferred example, the pH is about 7.0.

In one example, the yield of linalool production is at least 60 mg/L. For example, the yield of linalool production may be about 60 mg/L, about 100 mg/L, about 200 mg/L, about 300 mg/L, about 400 mg/L, about 500 mg/L, about 600 mg/L, about 700 mg/L, about 800 mg/L, about 900 mg/L, about 1000 mg/L, about 1100 mg/L, about 1200 mg/L, about 1300 mg/L, about 1400 mg/L, about 1500 mg/L, about 1600 mg/L, about 1700 mg/L, about 1800 mg/L, about 1900 mg/L, about 2000 mg/L, about 2100 mg/L, about 2200 mg/L, about 2300 mg/L, about 2400 mg/L, about 2500 mg/L, about 2600 mg/L, about 2700 mg/L, about 2800 mg/L, about 2900 mg/L, about 3000 mg/L, about 3100 mg/L, about 3200 mg/L, about 3300 mg/L, about 3400 mg/L, about 3500 mg/L, about 3600 mg/L, about 3700 mg/L, about 3800 mg/L, about 3900 mg/L and about 4000 mg/L. In a preferred example, the yield of linalool production is at least 3000 mg/L.

In one example, the yield of linalool production is determined by the concentration of linalool per OD₆₀₀. For example, the yield of linalool production may be about 30 mg/L/OD₆₀₀, about 40 mg/L/OD₆₀₀, about 50 mg/L/OD₆₀₀, about 60 mg/L/OD₆₀₀, about 70 mg/L/OD₆₀₀, about 80 mg/L/OD₆₀₀, about 90 mg/L/OD₆₀₀, about 100 mg/L/OD₆₀₀, about 110 mg/L/OD₆₀₀, about 120 mg/L/OD₆₀₀, about 130 mg/L/OD₆₀₀, about 140 mg/L/OD₆₀₀, about 150 mg/L/OD₆₀₀, about 160 mg/L/OD₆₀₀, about 170 mg/L/OD₆₀₀, about 180 mg/L/OD₆₀₀, about 190 mg/L/OD₆₀₀, about 200 mg/L/OD₆₀₀, about 210 mg/L/OD₆₀₀, about 220 mg/L/OD₆₀₀, about 230 mg/L/OD₆₀₀, about 240 mg/L/OD₆₀₀, about 250 mg/L/OD₆₀₀, about 260 mg/L/OD₆₀₀, about 270 mg/L/OD₆₀₀, about 280 mg/L/OD₆₀₀, about 290 mg/L/OD₆₀₀ and about 300 mg/L/OD₆₀₀. In a preferred example, the yield of linalool production is at least 240 mg/L/OD₆₀₀.

In one example, the carbon yield of linalool production is calculated as a ratio of the mass of linalool produced and the total mass of metabolizable carbon. For example, the carbon yield of linalool production may be about 0.5%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.5%, about 16.5%, about 17.0%, about 18.5%, about 19.0%, about 19.5% and about 20.0%. In a preferred example, the carbon yield of linalool production is at least 15%.

The method as described herein may be used to produce different forms of linalool. The different forms of linalool include enantiomers of linalool and are not limited to (R)-linalool and(S)-linalool. It will be generally understood that the list is not exhaustive, and the method may be used to produce different combinations of the various types of linalool.

In another aspect, the present invention refers to a kit producing linalool, wherein the kit comprises the host cell as described herein with instructions for use.

In some examples, the host cell in the kit may be dissolved in solution or lyophilized.

In some examples, the host cell may be preserved by deep freezing.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Materials & Methods

### Strain and Plasmid Construction

E. coli BL21 DE3 strain (CGSC #12504) from the Coli Genetic Stock Center (CGSC) of Yale university was used for monoterpenoid production. The gene tnaA was deleted from the genome of BL21 DE3 strain. A new series of plasmids were constructed by combining the operons hmgS-atoB-hmgR and mevk-pmK-pmd-idi into the same p15A-spec (L2-8) vector with three different promoter variants (TM1, TM2 and TM3). The new plasmid set included 3×3-9 plasmids (Table 1). The genes ApLS, linalool synthase from Agrocybe pediades and GPPS (e.g., ispA^{S80F} and AgGPPS) were cloned into a p15A-kan vector.

TABLE 1
Plasmid information

| | |
|---|---|
| | Promoters of |
| Serial | modules² |

| # | Plasmids¹ | M1³ | M2⁴ |
|---|---|---|---|
| 1 | sps001 | TM1 | TM1 |
| 2 | sps002 | TM1 | TM2 |
| 3 | sps003 | TM1 | TM3 |
| 4 | sps004 | TM2 | TM1 |
| 5 | sps005 | TM2 | TM2 |
| 6 | sps006 | TM2 | TM3 |
| 7 | sps007 | TM3 | TM1 |
| 8 | sps008 | TM3 | TM2 |
| 9 | sps009 | TM3 | TM3 |

¹sps001-009 expressing two modules M1 and M2.
²The relative strengths for the TM1, TM2, TM3 promoters were about 92%, 37% and 16%, respectively to that of the T7 promoter.
³The module M1 contains the three genes - HmgS, hmgR and atoB
⁴The module M2 contains the three genes - mevK, pmk, pmd, and idi

### Enzyme Truncation and Fusion

The C-terminal 19 amino acids were truncated from ApLS with an inhouse simple cloning method, and the truncation was further validated by DNA sequencing. The truncated ApLS was named ApLS*. The ApLS* was directly fused with ispA^{S80F} with two orientations: IspAS80F-ApLS* (N-terminal fusion of GPPS to ApLS*) and ApLS⁺-IspAS80F (C-terminal fusion of GPPS to ApLS*).

### Media and Culture Conditions

Excluding carbon sources, chemically defined medium (or defined medium) contained 2 g/L (NH4) 2SO4, 4.2 g/L KH2PO4, 11.24 g/L K2HPO4, 1.7 g/L citric acid, 0.5 g/L MgSO4 and 10 ml/L trace element solution, pH 7.0. The trace element solution (100X) contained 0.25 g/L CoCI2-6H2O, 1.5 g/L MnSO4-4H2O, 0.15 g/L CuSO4. 2H2O, 0.3 g/L H3BO3, 0.25 g/L Na2MoO4.2H2O, 0.8 g/L Zn (CH3COO) 2, 5 g/L Fe (III) citrate and 0.84 g/L EDTA, pH 8.0.

2xPY medium consisted of 20 g/L peptone, 10 g/L yeast extract and 10 g/L NaCl.

Excluding carbon sources, TB medium consisted of 12 g/L tryptone, 24 g/L yeast extract, 2.31 g/L KH2PO4, and 12.54 g/L K2HPO4.

For both defined and TB medium, two variations of carbon sources were used: the lactose auto-induction medium (LAM), in which the carbon sources contained 2 g/L glucose, 8 g/L glycerol and 10-50 mM lactose.; the IPTG manual-induction medium (IMM), in which the carbon source was 10 g/L glucose. In LAM, lactose was used as inducer. In IMM, IPTG served as inducer.

The engineered cells were grown in 1 mL of the above media in 14 mL BD Falcon™ tube at 28° C./250 rpm for 3 days. In addition, 200 μL (20% v/v) dodecane or isopropyl myristate was used to extract the terpenoids during cell culture. In IMM, cells were initially grown at 37° C./250 rpm until OD₆₀₀ reached 1-2, induced by 0.01˜0.25 mM IPTG, and were then grown at 28° C. for 3 days. In LAM, cells were grown at 28° C./250 rpm for 3 days and automatically induced by 10-50 mM lactose. The cultures of linalool cells were supplemented with antibiotics (50 μg/ml kanamycin and 50 μg/ml spectinomycin) to maintain the two plasmids.

### Quantification of Linalool by GC/MS

The linalool samples were prepared by performing a 100-10000× dilution of organic layer into hexane. The samples were analyzed on an Agilent 7890 gas chromatography equipped with an Agilent 5977B MSD. Samples were injected into an Agilent DB-5 column with a split ratio of 100:1 at 350° C. The oven program started at 120° C. for 1 min, was raised up to 170° C. at 10° C./min, then to 310° C. at 100° C./min and maintained at 310° C. for another 2 min. Mass spectrometer was operated in electron ionization (El) mode with full-scan analysis (m/z 30-300, 2 spectra/s). Linalool concentrations were quantified by interpolating with a standard curve prepared by their authentic standards (Sigma-Aldrich, Singapore).

### Results

### Optimization of the Terpenoid Pathway

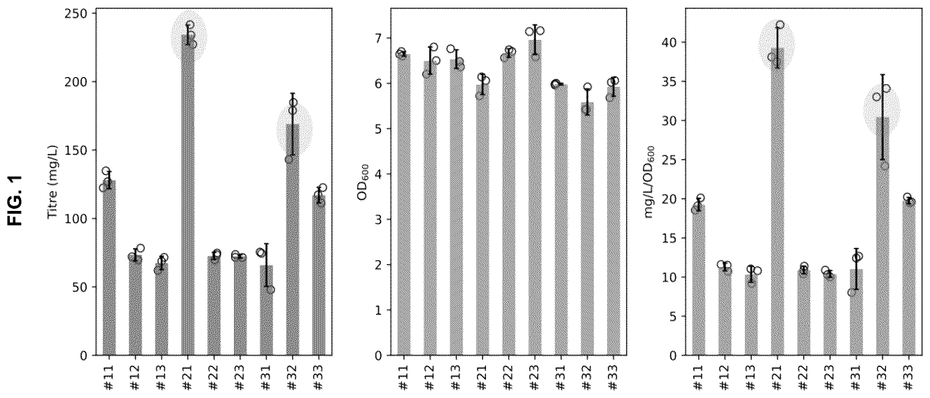

The linalool strain was built on top of previous geraniol strains. Briefly, by dividing the mevalonate pathway into two modules (upper module: including the genes atoB, hmgS and truncated hmgR; lower module: including the genes mevk, pmK, pmd and idi), the pathway was systematically balanced by promoter engineering (FIG. 1). The top two strains #21, #32 produced about 234.2 and 168.9 mg/L of linalool, respectively. The specific yields (titres normalized by OD₆₀₀) of linalool of the two strains were 39.3 and 30.4 mg/L/OD₆₀₀, respectively.

### Induction Timing and Inducer Concentrations

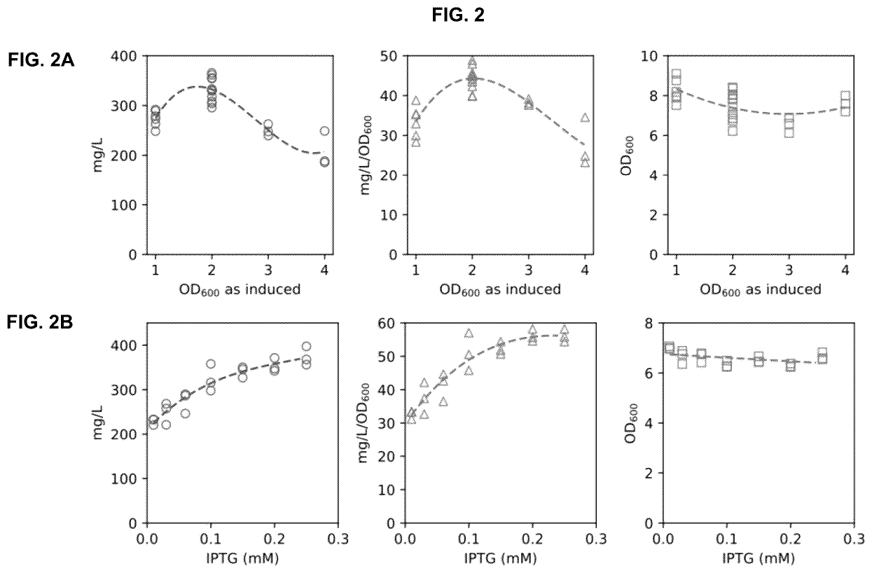

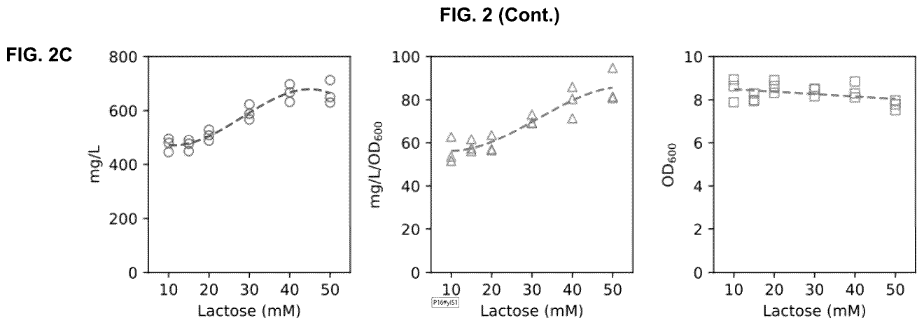

After the upstream mevalonate pathway design was fixed, two parameters were studied: the induction timing and inducer concentrations. Both parameters are known to be critical to produce secondary metabolites. Here, an auto-induction defined media (AIDM) was used to optimize the induction timing. AIDM contained three kinds of carbons: glucose, glycerol and lactose. All of them can serve as carbon sources for cell growth and linalool production. In addition, glucose also controls the time of induction by imposing catabolite repression on the uptake of lactose (the inducer) and glycerol. Glycerol serves the bridging carbon source while glucose is depleting, and the uptake of lactose is activating. Lactose serves as the inducer in AIDM, and 1 mole of lactose is converted to 1 mole of galactose (non-metabolizable) and 1 mole of glucose (metabolizable). The BL21 (DE3) strain in this study can use glucose but not galactose as carbon substrate, i.e., about half of lactose supplied can be used as carbon substrate. The linalool yield was observed to be highest (332.3 mg/L) when cells were induced at about OD₆₀₀ of 2 (FIG. 2A). Induction earlier or later resulted in the decrease in linalool production. Next, two inducers IPTG and lactose were compared and optimized. The production of linalool increased as more inducers (both IPTG and lactose) were used and eventually saturated (FIGS. 2B and C). For IPTG, linalool titre increased from 229 to 374 mg/L (32.7 to 56.2 mg/L/OD₆₀₀) and saturated as IPTG concentration increased to 0.2-0.25 mM. For lactose, the linalool titre increased from 472 to 665 mg/L (56.0 to 79.3 mg/L/OD₆₀₀) and saturated at 40-50 mM lactose.

### Enzyme Truncation, Fusion and Debottlenecking the Linalool Pathway

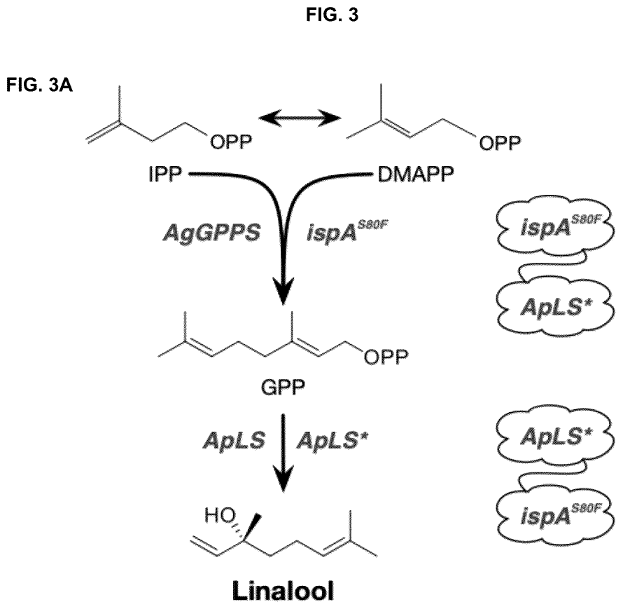

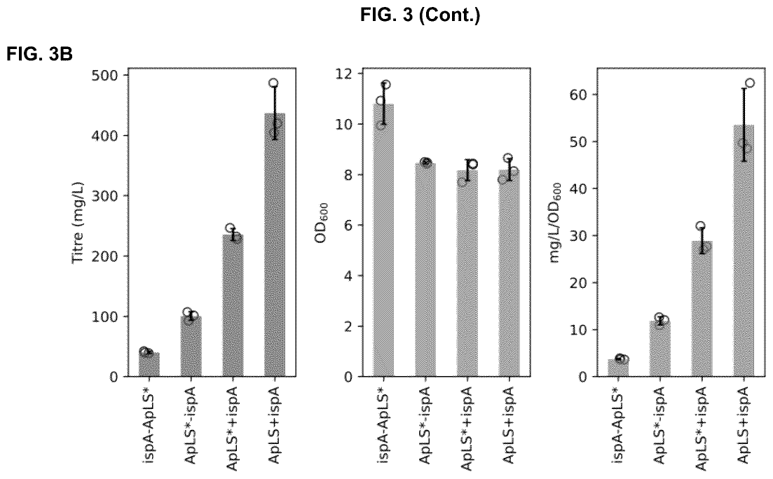

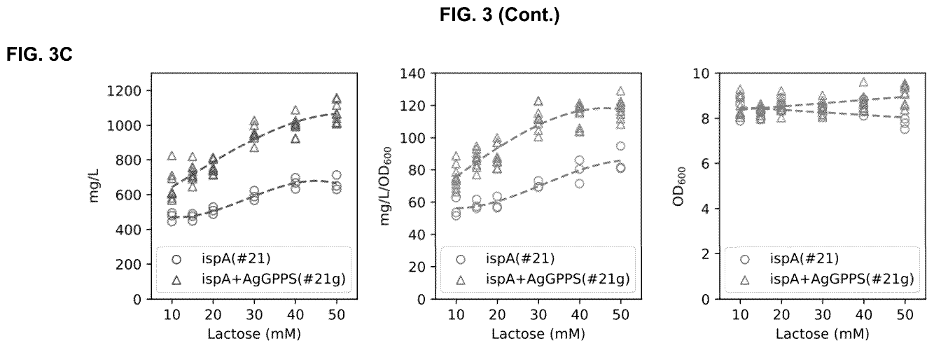

Next, the rate limiting step of the whole pathway for linalool bioproduction was identified. Previously, ˜81% of the theoretic yields for two sesquiterpenes (viridiflorol and amorpha-4,11-diene) using the mevalonate pathway were achieved. As the linalool strain used the same design and mevalonate pathway genes, the carbon fluxes from glucose to isoprenyl diphosphate (IPP) and (DMAPP) were assumed to be not limited. Instead, the last two enzymes that covert IPP/DMAPP to linalool: GPP synthase (GPPS, E. coli ispA^{S80F}) and linalool synthase (ApLS) were focused on. GPPS catalyses the formation of GPP from IPP and DMAPP. Linalool synthase transforms GPP to linalool. Previously, ApLS and its C-terminal truncated mutant (with the first 19 amino acids in C-terminal deleted, named ApLS*) were studied. The ApLS* remains very active and its expression in E. coli was about 5-10 times higher than that of the non-truncated ApLS. In addition, it was reported that the fusion of GPPS and monoterpene synthase could improve the yield of monoterpenes by channeling more IPP/DMAPP to monoterpenes. Therefore, the truncation and fusion strategy were studied (FIG. 3A). Two orientations of the fusion protein (left and right sides represent N- and C-terminal of the protein, respectively) were tested: one is ispA-ApLS⁺, the other is ApLS⁺-ispA (FIG. 3B). However, both the fusion designs produced less linalool than the non-fused free version (ApLS*+ispA, FIG. 3B). In addition, the strain using ApLS* unexpectedly produced less linalool as compared to that expressing the wildtype ApLS. The unsuccess of the two strategies led to the hypothesis that the bottleneck of linalool production was not linalool synthase but could be the insufficient activity of GPPS.

To test this hypothesis, another GPPS, the N-terminal truncated GPPS from Abies grandis (AgGPPS) was expressed in the best strain #21 and the resulting strain was named #21g. Indeed, the #21g strain produced about 36%-61% higher linalool than the #21 strain in all the lactose concentrates tested (FIG. 3C). Similar trend was observed between the linalool production and lactose concentrations for strains #21g and #21, and the linalool titre reached 1071 mg/L (118.2 mg/L/OD₆₀₀) with 40-50 mM lactose. The results supported that the supply of GPP was limiting in strain #21, and supplementation of additional GPPS could overcome the limitation and boosted markedly the linalool production.

### Medium Optimization for Linalool Production

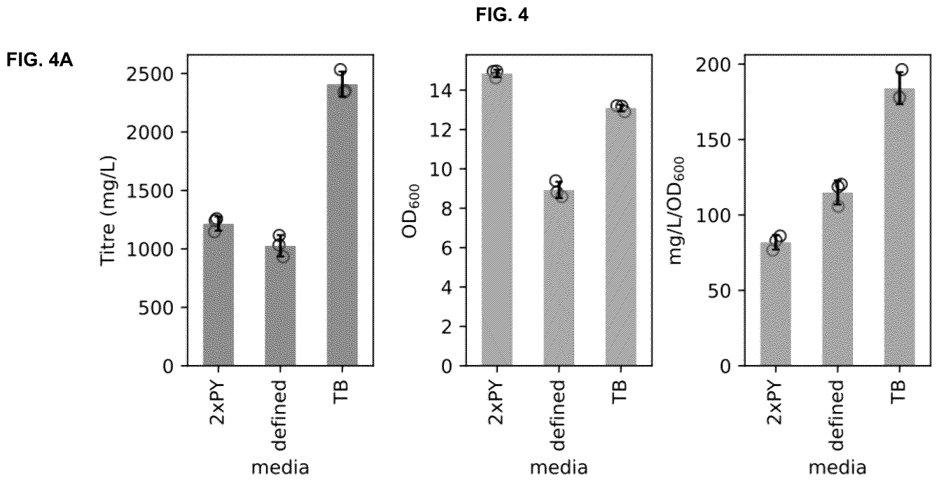

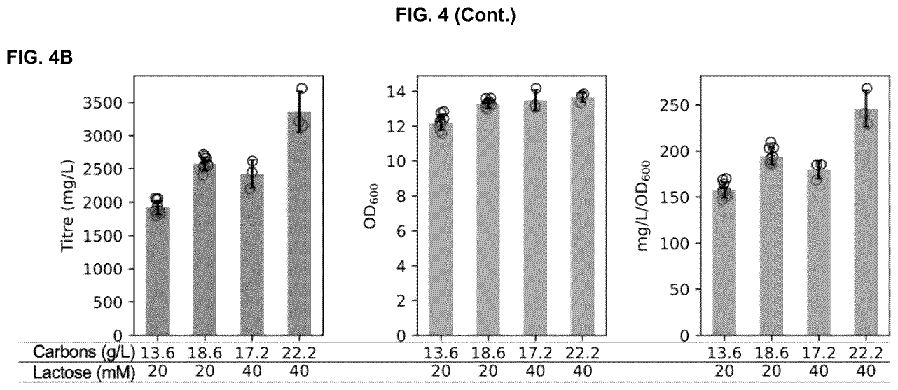

Furthermore, different media for linalool production were compared. Based on previous studies, three media: 2xPY, TB and defined media were chosen (FIG. 4A). The former two media are complex media and the last one is a chemically defined medium consisting of only salts and organic carbons. The titre (2049 mg/L) and specific yield (184.1 mg/L/OD₆₀₀) of linalool in TB medium were highest among the three media (FIG. 4A). The titres of linalool were similar for 2xPY and defined media. However, because of the low OD₆₀₀, the specific yield of linalool (114.9 mg/L/OD₆₀₀) in defined media was 40% higher than that (81.9 mg/L/OD₆₀₀) in 2xPY. Next, the concentrations of the three carbons, glucose, glycerol and lactose were further titrated. In the condition (3 g/L glucose, 12 g/L glycerol and 40 mM lactose), the strain #21g produced up to 3360 mg/L (or 246.1 mg/L/OD₆₀₀) of linalool.

### Linalool Production Yields (Yp/s)

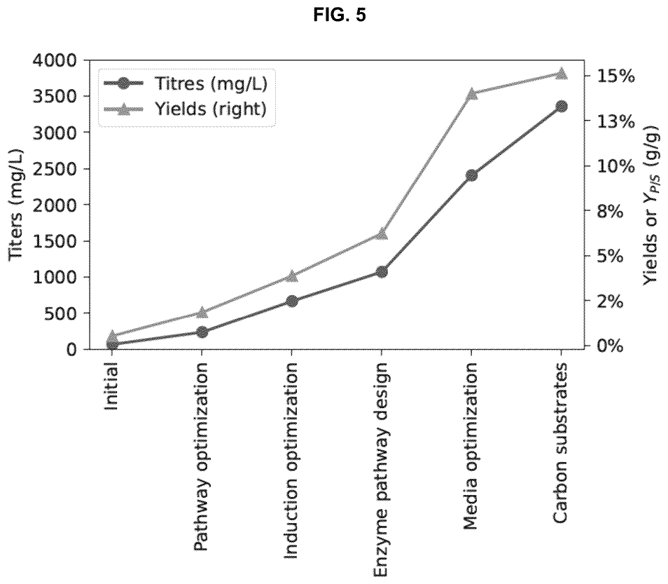

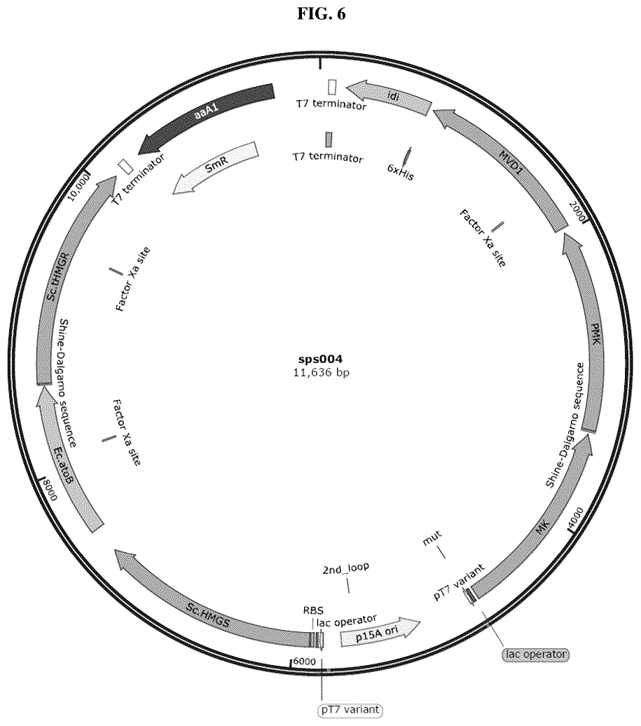

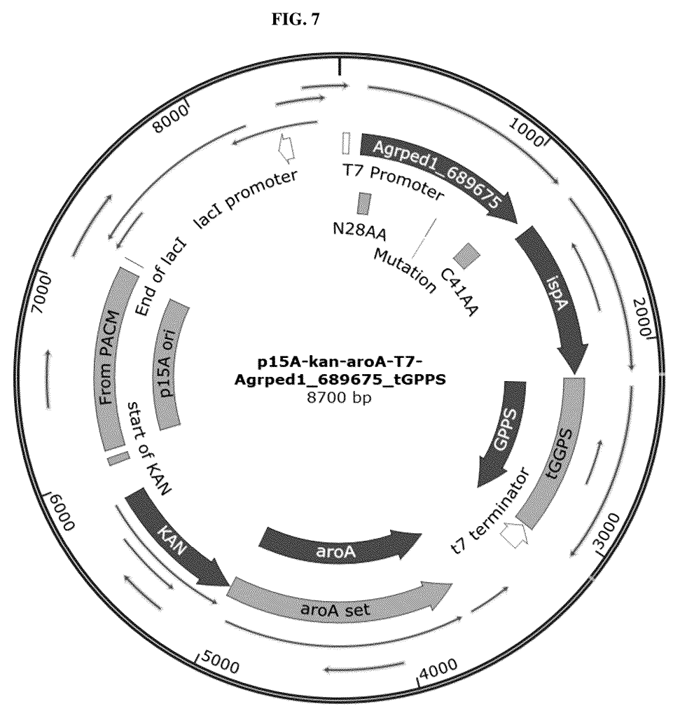

All the strategies used were summarized and the linalool yields were calculated (mass of linalool produced/mass of metabolizable carbons). Here, as the BL21 strain used cannot metabolize galactose, only half of lactose (mass) can be used as the carbon substrate. Hence, the total carbon was the sum of half mass of lactose, the total mass of glucose and glycerol, and linalool carbon yields were calculated accordingly (FIG. 5). With all the strategies combined, the titres were increased from 66 to 3360 mg/L and the yields from 0.5% to 15%. The yield is the highest among the current literature.

TABLE 2
Summary of sequence listing.

| Description | DNA/Protein sequence | SEQ ID NO |
|---|---|---|
| Nucleic acid | atgggtaacgtgttacaagccgggctggggcaaaatccggcg | SEQ ID NO: 1 |
| sequence of atoB | cgtcaggcactgttaaaaagcgggctggcagaaacggtgtgc | |
| gene | ggattcacggtcaataaagtatgtggttcgggtcttaaaagtgtg | |
| | gcgcttgccgcccaggccattcaggcaggtcaggcgcagag | |
| | cattgtggcggggggtatggaaaatatgagtttagccccctact | |
| | tactcgatgcaaaagcacgctctggttatcgtcttggagacgga | |
| | caggtttatgacgtaatcctgcgcgatggcctgatgtgcgccac | |
| | ccatggttatcatatggggattaccgccgaaaacgtggctaaa | |
| | gagtacggaattacccgtgaaatgcaggatgaactggcgcta | |
| | cattcacagcgtaaagcggcagccgcaattgagtccggtgctt | |
| | ttacagccgaaatcgtcccggtaaatgttgtcactcgaaagaa | |
| | aaccttcgtcttcagtcaagacgaattcccgaaagcgaattca | |
| | acggctgaagcgttaggtgcattgcgcccggccttcgataaag | |
| | caggaacagtcaccgctgggaacgcgtctggtattaacgacg | |
| | gtgctgccgctctggtgattatggaagaatctgcggcgctggca | |
| | gcaggccttacccccctggctcgcattaaaagttatgccagcg | |
| | gtggcgtgccccccgcattgatgggtatggggccagtacctgc | |
| | cacgcaaaaagcgttacaactggcggggctgcaactggcgg | |
| | atattgatctcattgaggctaatgaagcatttgctgcacagttcctt | |
| | gccgttgggaaaaacctgggctttgattctgagaaagtgaatgt | |
| | caacggggggccatcgcgctcgggcatcctatcggtgccag | |
| | tggtgctcgtattctggtcacactattacatgccatgcaggcacg | |
| | cgataaaacgctggggctggcaacactgtgcattggcggcgg | |
| | tcagggaattgcgatggtgattgaacggttgaattaa | |
| Nucleic acid | atgaaactctcaactaaactttgttggtgtggtattaaaggaaga | SEQ ID NO: 2 |
| sequence of hmgS | cttaggccgcaaaagcaacaacaattacacaatacaaacttg | |
| gene | caaatgactgaactaaaaaaacaaaagaccgctgaacaaa | |
| | aaaccagacctcaaaatgtcggtattaaaggtatccaaatttac | |
| | atcccaactcaatgtgtcaaccaatctgagctagagaaatttga | |
| | tggcgtttctcaaggtaaatacacaattggtctgggccaaacca | |
| | acatgtcttttgtcaatgacagagaagatatctactcgatgtccct | |
| | aactgttttgtctaagttgatcaagagttacaacatcgacaccaa | |
| | caaaattggtagattagaagtcggtactgaaactctgattgaca | |
| | agtccaagtctgtcaagtctgtcttgatgcaattgtttggtgaaaa | |
| | cactgacgtcgaaggtattgacacgcttaatgcctgttacggtg | |
| | gtaccaacgcgttgttcaactctttgaactggattgaatctaacg | |
| | catgggatggtagagacgccattgtagtttgcggtgatattgcc | |
| | atctacgataagggtgccgcaagaccaaccggtggtgccggt | |
| | actgttgctatgtggatcggtcctgatgctccaattgtatttgactct | |
| | gtaagagcttcttacatggaacacgcctacgatttttacaagcc | |
| | agatttcaccagcgaatatccttacgtcgatggtcatttttcattaa | |
| | cttgttacgtcaaggctcttgatcaagtttacaagagttattccaa | |
| | gaaggctatttctaaagggttggttagcgatcccgctggttcgg | |
| | atgctttgaacgttttgaaatatttcgactacaacgttttccatgttc | |
| | caacctgtaaattggtcacaaaatcatacggtagattactatat | |
| | aacgatttcagagccaatcctcaattgttcccagaagttgacgc | |
| | cgaattagctactcgcgattatgacgaatctttaaccgataaga | |
| | acattgaaaaaacttttgttaatgttgctaagccattccacaaag | |
| | agagagttgcccaatctttgattgttccaacaaacacaggtaac | |
| | atgtacaccgcatctgtttatgccgcctttgcatctctattaaacta | |
| | tgttggatctgacgacttacaaggcaagcgtgttggtttattttctt | |
| | acggttccggtttagctgcatctctatattcttgcaaaattgttggtg | |
| | acgtccaacatattatcaaggaattagatattactaacaaatta | |
| | gccaagagaatcaccgaaactccaaaggattacgaagctgc | |
| | catcgaattgagagaaaatgcccatttgaagaagaacttcaa | |
| | acctcaaggttccattgagcatttgcaaagtggtgtttactacttg | |
| | accaacatcgatgacaaatttagaagatcttacgatgttaaaa | |
| | aataa | |
| Nucleic acid | atgagcttaccgttcctgacttcggcaccgggcaaagttatcatt | SEQ ID NO: 3 |
| sequence of mevk | ttcggcgagcactctgctgtttacaacaaaccggcagttgcgg | |
| gene | cctccgtatctgcactgcgcacttatctgctgatctctgaaagctc | |
| | cgccccggatactattgaactggactttccggacatttcctttaac | |
| | cacaaatggagcattaacgactttaacgcgatcactgaagatc | |
| | aggtaaactcccagaaactggcaaaagcacagcaggctac | |
| | cgatggtctgagccaggaactggtgtccctcctcgatcctttgct | |
| | ggctcaactctcggaatcgttccattaccatgctgctttctgttttct | |
| | gtatatgtttgtttgcctctgcccgcacgcgaaaaacatcaaatt | |
| | ctctctgaaatcgactctgccgattggtgccggcctgggttcgtc | |
| | cgcatctatttccgtttccctggcgctggccatggcctatctgggc | |
| | ggtctgatcggttccaacgacctggaaaaactctcggaaaac | |
| | gataagcacatcgttaaccagtgggcgttcatcggtgaaaaat | |
| | gtatccacggtaccccatccggtatcgataatgcggttgctacct | |
| | acggtaacgcgttactgttcgaaaaagattctcataacggtact | |
| | atcaacactaacaacttcaaatttttggacgattttccggcgattc | |
| | cgatgatcctgacttacacccgcatcccgcgtagcaccaagg | |
| | atctggttgcacgcgttcgtgtactcgtgaccgaaaaattcccg | |
| | gaggttatgaaaccgatcctggatgcaatgggtgagtgcgcg | |
| | ctgcagggattagaaatcatgaccaaactgtcgaagtgtaaa | |
| | ggtacggacgacgaagctgttgaaacaaacaacgaactgta | |
| | tgaacagctgctggaactgatccgtatcaaccacggcctgctg | |
| | gtcagcattggtgtgagccacccgggcctggaactgattaaaa | |
| | atctttcggatgacctgcgcattggttctaccaagctgactggtg | |
| | ctggcggcggtggctgttctctgaccctcctgcgtcgcgatatta | |
| | cccaggaacaaatcgactcgttcaaaaaaaaactgcaggat | |
| | gacttttcttatgaaaccttcgaaaccgacctgggcggtaccgg | |
| | ctgttgtctcctgtccgccaaaaacttgaacaaagatctgaaaa | |
| | tcaaatctctcgtotttcagctgtttgaaaacaaaactaccacca | |
| | aacaacaaattgatgacctgctgctcccgggcaacaccaactt | |
| | accgtggacttcctaa | |
| Nucleic acid | atgtctgagcttcgcgctttctccgctccgggcaaggccctgctg | SEQ ID NO: 4 |
| sequence of pmK | gccggaggctatctggtgctggacaccaaatatgaagcgtttgt | |
| gene | agttggtctgtctgcccgtatgcatgcggtcgcgcacccgtatg | |
| | ggtcgctgcagggttcagataaattcgaggtgcgagtgaaaa | |
| | gcaaacagtttaaagacggtgagtggctgtatcacattagccc | |
| | gaaatctggttttatcccggtatccatcggcggttccaaaaacc | |
| | cgttcattgaaaaagttattgccaacgttttctcctattttaaaccta | |
| | acatggatgactactgtaaccgtaacctgttcgtgattgatattttc | |
| | tctgacgatgcttaccattcccaggaagacagcgttacggaac | |
| | accgtggcaaccgtcgtctgtcgtttcattcccaccgtatcgaag | |
| | aagttccgaagactggcctgggtagctctgcaggcctggttac | |
| | cgtcctgactactgctctggcctctttttttgtgtoggatctggaaa | |
| | acaacgttgacaaatatcgtgaggtaattcataacctggctcag | |
| | gtcgcacactgccaggcgcagggcaaaatcggctccggtttc | |
| | gatgttgctgcggcagcttatggctccattegttaccgtogcttcc | |
| | cgcctgctctgatctcaaacctgccggatattggtagcgcaacc | |
| | tacggatcgaagctggctcacctggtggatgaggaagattgg | |
| | aatatcaccattaaatctaaccacctgccgtctggcctgaccct | |
| | gtggatgggtgatatcaaaaacggctctgaaaccgtcaaact | |
| | ggtacagaaagttaagaattggtatgattctcacatgccggaat | |
| | ccctgaaaatctacaccgagctggatcacgcgaactcacgttt | |
| | catggacggtctgtccaaactggaccgtctgcacgaaaccca | |
| | cgatgattacagcgaccagatcttcgaatctctggaacgtaac | |
| | gactgcacctgtcaaaaatacccggaaatcaccgaagttcgt | |
| | gacgcggtagcgaccatccgccgctotttccgtaaaatcacta | |
| | aggaaagcggcgctgacatcgaaccgccggttcagacctcc | |
| | ctgctggacgattgccagactctgaaaggggtgctgacctgtct | |
| | gattccgggtgcgggtggttatgacgctatcgcagtgatcacga | |
| | aacaggatgtagacctgcgtgcgcagactgcaaacgacaaa | |
| | cgttttagcaaagtacaatggctggatgttacccaggcggattg | |
| | gggtgttcgtaaagaaaaggaccctgaaacctacctggataa | |
| | ataa | |
| Nucleic acid | atgaccgtttacacagcatccgttaccgcacccgtcaacatcg | SEQ ID NO: 5 |
| sequence of pmd | caacccttaagtattgggggaaaagggacacgaagttgaatc | |
| gene | tgcccaccaattcgtccatatcagtgactttatcgcaagatgacc | |
| | tcagaacgttgacctctgcggctactgcacctgagtttgaacgc | |
| | gacactttgtggttaaatggagaaccacacagcatcgacaatg | |
| | aaagaactcaaaattgtctgcgcgacctacgccaattaagaa | |
| | aggaaatggaatcgaaggacgcctcattgcccacattatctca | |
| | atggaaactccacattgtctccgaaaataactttcctacagcag | |
| | ctggtttagcttcctccgctgctggctttgctgcattggtctctgcaa | |
| | ttgctaagttataccaattaccacagtcaacttcagaaatatccc | |
| | gtatagcaagaaaggggtctggttcagcttgtagatcgttgtttg | |
| | gcggatacgtggcctgggaaatgggaaaagctgaagatggt | |
| | catgattocatggcagtacaaatcgcagacagctctgactggc | |
| | ctcagatgaaagcttgtgtcttagtcgtcagcgatattaaaaag | |
| | gatgtgagttccactcagggtatgcaattgaccgtggcaacctc | |
| | cgaactatttaaagaaagaattgaacatgtcgtaccaaagag | |
| | atttgaagtcatgcgtaaagccattgttgaaaaagatttcgcca | |
| | cctttgcaaaggaaacaatgatggattccaactctttccatgcc | |
| | acatgtttggactotttccctccaatattctacatgaatgacacttc | |
| | caagcgtatcatcagttggtgccacaccattaatcagttttacgg | |
| | agaaacaatcgttgcatacacgtttgatgcaggtccaaatgctg | |
| | tgttgtactacttagctgaaaatgagtcgaaactctttgcatttatc | |
| | tataaattgtttggctctgttcctggatgggacaagaaatttacta | |
| | ctgagcagcttgaggctttcaaccatcaatttgaatcatctaactt | |
| | tactgcacgtgaattggatottgagttgcaaaaggatgttgcca | |
| | gagtgattttaactcaagtcggttcaggcccacaagaaacaaa | |
| | cgaatctttgattgacgcaaagactggtctaccaaaggaataa | |
| Nucleic acid | atgcatcatcatcaccatcacgagctccaaacggaacacgtc | SEQ ID NO: 6 |
| sequence of idi | attttattgaatgcacagggagttcccacgggtacgctggaaa | |
| gene | agtatgccgcacacacggcagacacccgcttacatctcgcgtt | |
| | ctccagttggctgtttaatgccaaaggacaattattagttacccg | |
| | ccgcgcactgagcaaaaaagcatggcctggcgtgtggacta | |
| | actcggtttgtgggcacccacaactgggagaaagcaacgaa | |
| | gacgcagtgatccgccgttgccgttatgagcttggcgtggaaat | |
| | tacgcctcctgaatctatctatcctgactttcgctaccgcgccacc | |
| | gatccgagtggcattgtggaaaatgaagtgtgtccggtatttgc | |
| | cgcacgcaccactagtgcgttacagatcaatgatgatgaagtg | |
| | atggattatcaatggtgtgatttagcagatgtattacacggtattg | |
| | atgccacgccgtgggcgttcagtccgtggatggtgatgcaggc | |
| | gacaaatcgcgaagccagaaaacgattatctgcatttaccca | |
| | gcttaaataa | |
| Nucleic acid | atggttttaaccaataaaacagtcatttctggatcgaaagtcaa | SEQ ID NO: 7 |
| sequence of | aagtttatcatctgcgcaatcgagctcatcaggaccttcatcatc | |
| truncated hmgR | tagtgaggaagatgattcccgcgatattgaaagcttggataag | |
| gene | aaaatacgtcctttagaagaattagaagcattattaagtagtgg | |
| | aaatacaaaacaattgaagaacaaagaggtcgctgccttggt | |
| | tattcacggtaagttacctttgtacgctttggagaaaaaattaggt | |
| | gatactacgagagcggttgcggtacgtaggaaggctctttcaa | |
| | ttttggcagaagctcctgtattagcatctgatcgtttaccatataaa | |
| | aattatgactacgaccgcgtatttggcgcttgttgtgaaaatgtta | |
| | taggttacatgcctttgcccgttggtgttataggccccttggttatc | |
| | gatggtacatcttatcatataccaatggcaactacagagggttg | |
| | tttggtagcttctgccatgcgtggctgtaaggcaatcaatgctgg | |
| | cggtggtgcaacaactgttttaactaaggatggtatgacaaga | |
| | ggcccagtagtccgtttcccaactttgaaaagatctggtgcctgt | |
| | aagatatggttagactcagaagagggacaaaacgcaattaa | |
| | aaaagcttttaactctacatcaagatttgcacgtctgcaacatatt | |
| | caaacttgtctagcaggagatttactcttcatgagatttagaaca | |
| | actactggtgacgcaatgggtatgaatatgatttctaaaggtgtc | |
| | gaatactcattaaagcaaatggtagaagagtatggctgggaa | |
| | gatatggaggttgtctccgtttctggtaactactgtaccgacaaa | |
| | aaaccagctgccatcaactggatcgaaggtcgtggtaagagt | |
| | gtcgtcgcagaagctactattcctggtgatgttgtcagaaaagt | |
| | gttaaaaagtgatgtttccgcattggttgagttgaacattgctaag | |
| | aatttggttggatctgcaatggctgggtctgttggtggatttaacg | |
| | cacatgcagctaatttagtgacagctgttttcttggcattaggaca | |
| | agatcctgcacaaaatgttgaaagttccaactgtataacattga | |
| | tgaaagaagtggacggtgatttgagaatttccgtatccatgcca | |
| | tccatcgaagtaggtaccatcggtggtggtactgttctagaacc | |
| | acaaggtgccatgttggacttattaggtgtaagaggcccgcat | |
| | gctaccgctcctggtaccaacgcacgtcaattagcaagaata | |
| | gttgcctgtgccgtcttggcaggtgaattatccttatgtgctgccct | |
| | agcagccggccatttggttcaaagtcatatgacccacaacag | |
| | gaaacctgctgaaccaacaaaacctaacaatttggacgcca | |
| | ctgatataaatcgtttgaaagatgggtccgtcacctgcattaaat | |
| | cctaa | |
| Polypeptide | MVLTNKTVISGSKVKSLSSAQSSSSGPSSSS | SEQ ID NO: 8 |
| sequence of | EEDDSRDIESLDKKIRPLEELEALLSSGNTKQ | |
| truncated hmgR | LKNKEVAALVIHGKLPLYALEKKLGDTTRAVA | |
| gene | VRRKALSILAEAPVLASDRLPYKNYDYDRVF | |
| | GACCENVIGYMPLPVGVIGPLVIDGTSYHIPM | |
| | ATTEGCLVASAMRGCKAINAGGGATTVLTKD | |
| | GMTRGPVVRFPTLKRSGACKIWLDSEEGQN | |
| | AIKKAFNSTSR | |
| | FARLQHIQTCLAGDLLFMRFRTTTGDAMGM | |
| | NMISKGVEYSLKQMVEEYGWEDMEVVSVSG | |
| | NYCTDKKPAAINWIEGRGKSVVAEATIPGDV | |
| | VRKVLKSDVSALVELNIAKNLVGSAMAGSVG | |
| | GFNAHAANLVTAVFLALGQDPAQNVESSNCI | |
| | TLMKEVDGDLRISVSMPSIEVGTIGGGTVLEP | |
| | QGAMLDLLGVRGPHATAPGTNARQLARIVA | |
| | CAVLAGELSLCAALAAGHLVQSHMTHNRKP | |
| | AEPTK PNNLDATDINRLKDGSVTCIKS | |
| Nucleic acid | AAAGAACAGCTGCAGGTATCCATGAAGGC | SEQ ID NO: 9 |
| sequence of | A | |
| C10AA_tGPPS | | |
| Nucleic acid | CGCCCGCTGGACCCGGCGTACGTGACCAA | SEQ ID NO: 10 |
| sequence of | AGAACAGCTGCAGGTATCCATGAAGGCA | |
| C19AA_tGPPS | | |
| Polypeptide | MFDFNKYMDSKAMTVNEALNKAIPLRYPQKI | SEQ ID NO: 11 |
| sequence of | YESMRYSLLAGGKRVRPVLCIAACELVGGTE | |
| truncated ApLS | ELAIPTACAIEMIHTMSLMHDDLPCIDNDDLR | |
| | RGKPTNHKIFGEDTAVTAGNALHSYAFEHIAV | |
| | STSKTVGADRILRMVSELGRATGSEGVMGG | |
| | QMVDIASEGDPSIDLQTLEWIHIHKTAMLLEC | |
| | SVVCGAIIGGASEIVIERARRYARCVGLLFQV | |
| | VDDILDVTKSSDELGKTAGKDLISDKATYPKL | |
| | MGLEKAKEFSDELLNRAKGELSCFDPVKAAP | |
| | LLGLADYVAFRQN | |
| Polypeptide | MDFPQQLEACVKQANQALSRFIAPLPFQNTP | SEQ ID NO: 12 |
| sequence of | VVETMQYGALLGGKRLRPFLVYATGHM | |
| IspA^{S80F} | FGVSTNTLDAPAAAVECIHAYFLIHDDLPAMD | |
| | DDDLRRGLPTCHVKFGEANAILAGDALQTLA | |
| | FSILSDADMPEVSDRDRISMISELASASGIAG | |
| | MCGGQALDLDAEGKHVPLDALERIHRHKTG | |
| | ALIRAAVRLGALSAGDKGRRALPVLDKYAESI | |
| | GLAFQVQDDILDVVGDTATLGKRQGADQQL | |
| | GKSTYPALLGLEQARKKARDLIDDARQSLKQ | |
| | LAEQSLDTSALEALADYIIQRNK | |
| Polypeptide | AYSAMATMGYNGMAASCHTLHPTSPLKPFH | SEQ ID NO: 13 |
| sequence of | GASTSLEAFNGEHMGLLRGYSKRKLSSYKN | |
| truncated AgGPPS | PASRSS | |
| | NATVAQLLNPPQKGKKAVE | |
| Nucleic acid | taatacgactcACTATAggggaattgtgagc | SEQ ID NO: 14 |
| sequence of T7 | | |
| promoter | | |
| Nucleic acid | aaattaatacgactcactaatggggaattgtgagcggataaca | SEQ ID NO: 15 |
| sequence of TM1 | | |
| promoter | | |
| Nucleic acid | aaattaatacgactcactcgaggggaattgtgagcggataac | SEQ ID NO: 16 |
| sequence of TM2 | a | |
| promoter | | |
| Nucleic acid | aaattaatacgactcactataaaggaattgtgagcggataaca | SEQ ID NO: 17 |
| sequence of TM3 | | |
| promoter | | |
| Nucleic acid | aaattaatacgactcactaTAGGGgaattgtgagcggataa | SEQ ID NO: 18 |
| sequence of TV1 | ca | |
| promoter | | |
| Nucleic acid | aaattaatacgactcactaCAGACgaattgtgagcggataa | SEQ ID NO: 19 |
| sequence of TV2 | ca | |
| promoter | | |
| Nucleic acid | aaattaatacgactcactaGCGGAgaattgtgagcggata | SEQ ID NO: 20 |
| sequence of TV3 | aca | |
| promoter | | |
| Nucleic acid | aaattaatacgactcactaacaccgaattgtgagcggataaca | SEQ ID NO: 21 |
| sequence of TV4 | | |
| promoter | | |
| Nucleic acid | taatacgactcX₁X₂X₃X₄X₅X₆ggggaattgtgagc, | SEQ ID NO: 22 |
| sequence of variant | wherein ‘X₁-X₆’ represents A or T or C or | |
| T7 promoter | G | |
| Polynucleotide | tttgtttaactttaAgaagGagatatacat | SEQ ID NO: 23 |
| sequence of RBS | | |
| for ispA_S80F | | |
| Polynucleotide | gagatataataacgagataagGaaAagacaaa | SEQ ID NO: 24 |
| sequence of RBS | | |
| for AgGPPS | | |
| Variant RBS | tttgtttaactttaX₁gaagX₂agatatacat, wherein | SEQ ID NO: 25 |
| sequence of | ‘X₁-X₂’ represents A or T or C or G | |
| ispA_S80F | | |
| Variant RBS | gagatataataacgagataagX₁aaX₂agacaaa, | SEQ ID NO: 26 |
| sequence of | wherein ‘X₁-X₂’ represents A or T or C or G | |
| AgGPPS | | |
| Polypeptide | PLAQWPWPRKLNQYYAEVKPESDQWIHGF | SEQ ID NO: 27 |
| sequence of linalool | GALDPKSQRSFDLCNFSLLG | |
| synthase, g9127 | SLVYPLLDKDGVRVGCDLMVLFFIYDEFTDK | |
| (Organism: | VDGDGARVYAEMVMDAIRD | |
| Lactarius | PHKERPQGEPKLGEITRQFWLRAMKVSSAE | |
| deliciosus) | AQRRFITTFAEYVYAVIDEA | |
| | SDRANGRVRGVEDYLKLTRLTAGGYPSFLAA | |
| | EAGLNIPDEVMAHPALQTIL | |
| | SLAAESLVLTNDMYSYNIEQASGHGGHNIVT | |
| | VIMNEKGVDLDGALNWLAE | |
| | YHGQVLSNFQAQHRLLPSWGPEVDADVGAF | |
| | VERLAYWIRGIDCWSLETE | |
| | RYFGTKGPEIKEHRRVTLLPKVKKPDVTPMM | |
| | AQLNA | |
| Polypeptide | MSSTQFIIPDLLVNWPWLRVIDPNLQQVTDE | SEQ ID NO: 28 |
| sequence of linalool | ANEWVESLDLFDPSQFKKF | |
| synthase, Psicya1 | KGCDFNRLGALVGHLQGKEHLRISCDLMNFY | |
| (Organism: | FAFDEYTDLADKDEAMKIS | |
| Psilocybe | KDVMNTFKHTEVPFDNKLIEMARQFFKRTID | |
| cyanescens) | VVGEDNPGFERFIADFDAYT | |
| | RSIIQEADDRDEGYIRSVDEYFILRRDTCGAK | |
| | PSFSFHGLGLRIPNEVFEHP | |
| | LVISMLEGATDLIAITNDMHSYGLEYSRGLDG | |
| | HNVITAIMKEYEVDLQAALY | |
| | WLSGYATKTISKFLTDRRKLPSWGPEIDVAV | |
| | HEFFERVGRCVRGYDAWS | |
| | YETNRYYGKNGLKIQETKRITLQPRDGAYITK | |
| | ERLQSSLA | |
| Polypeptide | MPRQYVIPDLLITWPWPRAINSSLTEVDEEAN | SEQ ID NO: 29 |
| sequence of linalool | AWVQSLDLFDSAQFKKFK | |
| synthase, Psicya2 | ACNFNLLGALVGPLRSKEHLRISCDLMNFYF | |
| (Organism: | AFDEYTDLANREEAIKIAKDV | |
| Psilocybe | MHAFRDTATPSDSKLIEMSRQFFRRTVDVVG | |
| cyanescens) | DDKPGFERFIADFDAYTTSI | |
| | IQEADDRAAGHIRNVEDYFILRRDTCGAKPSF | |
| | SFYALGLNIPDDVFENPLVI | |
| | SMLEGATDLIAITNDMHSYGLEHSRGLDGHN | |
| | VITAIMKEYNLDLQGALYWL | |
| | SGYATKTIAKFVSDRKKLPSWSPSVDAGVRE | |
| | FFDLVGRCVRGYDAWSYE | |
| | TKRYYGDKGLKIQKTRRITLQPRDAAYITKEQ | |
| | LKVSIAA | |
| Polypeptide | MSQSQFTIPDLLASWPWPRAKNPALDQNLE | SEQ ID NO: 30 |
| sequence of linalool | DEANAWVASLELFEPRQLD | |
| synthase, QDF5931 | KFKACQFNLLASLVGPIEGRDSLRISCDLMNF | |
| 5 (Organism: | YFAFDEYTDVVSGDEVMMI | |
| Hypholoma | VADVIQAFRDRESPEGSSKIKEMARQFFQRTI | |
| fasciculare) | ALVGEDTQGIDHFIADFEA | |
| | YAKSVVQEADDRVQGIVRNVEEYFILRRDTC | |
| | GGKPSFSFFGLGLCIPKEVF | |
| | DHPVMQSLTESATDLIAMINDMHSYALEHAR | |
| | GLDGHNVITAIMHEHSVDLQ | |
| | GAFYWLSGHASKTVSKFLNDRKNLPSWGSD | |
| | IDKAVNEYIDRMARCVRGY | |
| | DAWSYETNRYYGKNGLEVQKSRKIMLQHRE | |
| | LEMGYITRDQLLIGAA | |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A host cell comprising one or more vectors comprising a polynucleotide sequence encoding:
a) mevalonate pathway genes; and
b) linalool pathway genes,
wherein the linalool pathway genes comprise more than one diphosphate synthase gene, prenyltransferase gene or combinations thereof, and at least one linalool synthase gene.

2. The host cell according to claim 1, wherein
a) the mevalonate pathway genes are encoded by one or more polynucleotide sequences on one or more vectors; and
b) the linalool pathway genes are encoded by one or more polynucleotide sequences on one or more vectors, or wherein the linalool pathway genes are inserted into the genome of the host cell.

3. The host cell according to claim 1, wherein the mevalonate pathway genes are selected from the group consisting of atoB, hmgS, hmgR, mevK, pmK, pmd and idi, and the more than one diphosphate synthase gene, prenyltransferase gene or combinations thereof is a geranyl pyrophosphate synthase (GPPS) or a farnesyl pyrophosphate synthase (FPPS) gene, optionally, wherein the hmgR gene is truncated.

4. (canceled)

5. The host cell according to claim 1, wherein the linalool synthase gene is isolated from a eukaryote, optionally, wherein the eukaryote is a fungus selected from the group consisting of Agrocybe pediades, Galerina marginate, Hypholoma sublateritium, Hebeloma cylindrosporum, Lactarius deliciosus, Psilocybe cyanescens, Hypholoma fasciculare, Dictyostelium discoideum, Aspergillus calidoustus, Puccinia graminis f. sp. tritici, Melampsora larici-populina, Physarum polycephalum, Paxillus rubicundulus, and Postia placenta.

6. (canceled)

7. The host cell according to claim 5, wherein the linalool synthase gene is isolated from Agrocybe pediades (ApLS).

8. The host cell according to claim 7, wherein the ApLS is truncated at the C-terminal, optionally wherein the ApLS comprises the polypeptide sequence as set forth in SEQ ID NO. 11.

9. The host cell according to claim 3, wherein the more than one GPPS or FPPS gene is isolated from a prokaryote, a eukaryote, a plant, or combinations thereof.

10. The host cell according to claim 9, wherein at least one of the GPPS or FPPS gene is isolated from Escherichia coli, optionally wherein the FPPS gene isolated from Escherichia coli is a farnesyl pyrophosphate synthase mutated at one amino acid position, optionally wherein the mutated farnesyl pyrophosphate synthase is IspAS8OF comprising the polypeptide sequence as set forth in SEQ ID NO. 12.

11-12. (canceled)

13. The host cell according to claim 9, wherein at least one of the GPPS or FPPS gene is isolated from Abies grandis (AgGPPS), optionally wherein the GPPS isolated from Abies grandis (AgGPPS) is truncated at the N-terminal, optionally wherein the truncated AgGPPS comprises the polypeptide sequence as set forth in SEQ ID NO.: 13.

14-15. (canceled)

16. The host cell according to claim 1, wherein the polynucleotide sequence in each of the vectors is operably linked to one or more inducible promoters, wherein the one or more inducible promoter is a wild-type T7 RNA polymerase promoter or a variant of the wild-type T7 RNA polymerase promoter.

17. The host cell according to claim 3, wherein the more than one GPPS or FPPS is titrated by ribosomal binding site (RBS) engineering, optionally wherein the RBS comprises the polynucleotide sequence as set forth in SEQ ID NO: 23 or SEQ ID NO: 24.

18. (canceled)

19. The host cell according claim 1, wherein the host cell is deficient in at least one gene involved in amino acid degradation, optionally wherein the at least one gene involved in amino acid degradation is tnaA.

20. (canceled)

21. The host cell according to claim 1, wherein the host cell is a bacterial cell, optionally wherein the bacterial cell is an Escherichia coli cell.

22. (canceled)

23. A method of producing linalool comprising culturing the host cell according to claim 1 in a culture medium, wherein the culture medium optionally comprises an inducer and at least one carbon substrate.

24. The method according to claim 23, wherein the inducer is lactose or IPTG, and the at least one carbon substrate is selected from the group consisting of acetate, glycerol, glucose and lactose, and combinations thereof.

25. The method according to claim 23, wherein a titre of linalool production is at least 3000 mg/L.

26. The method according to claim 23, wherein a yield of linalool production is determined by a concentration of linalool per OD₆₀₀, and wherein the yield of linalool production is at least 240 mg/L/OD 600.

27. The method according to claim 23, wherein a carbon yield of linalool production is at least 15% of a total carbon mass.

28. A kit for producing linalool, wherein the kit comprises the host cell of claim 1 with instructions for use.

29. The kit according to claim 28, wherein the host cell is dissolved in solution, lyophilized, or preserved by deep freezing.

30. (canceled)


SEQUENCE LISTING (30 sequences)

SEQ ID NO: 1 - DNA, 1029 bp, Escherichia coli
  source 1..1029: mol_type = genomic DNA
atgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 2 - DNA, 1476 bp, synthetic construct
  source 1..1476: mol_type = other DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 3 - DNA, 1332 bp, synthetic construct
  source 1..1332: mol_type = other DNA
atgagcttaccgttcctgacttcggcaccgggcaaagttatcattttcggcgagcactctgctgtttacaacaaaccggcagttgcggcctccgtatctgcactgcgcacttatctgctgatctctgaaagctccgccccggatactattgaactggactttccggacatttcctttaaccacaaatggagcattaacgactttaacgcgatcactgaagatcaggtaaactcccagaaactggcaaaagcacagcaggctaccgatggtctgagccaggaactggtgtccctcctcgatcctttgctggctcaactctcggaatcgttccattaccatgctgctttctgttttctgtatatgtttgtttgcctctgcccgcacgcgaaaaacatcaaattctctctgaaatcgactctgccgattggtgccggcctgggttcgtccgcatctatttccgtttccctggcgctggccatggcctatctgggcggtctgatcggttccaacgacctggaaaaactctcggaaaacgataagcacatcgttaaccagtgggcgttcatcggtgaaaaatgtatccacggtaccccatccggtatcgataatgcggttgctacctacggtaacgcgttactgttcgaaaaagattctcataacggtactatcaacactaacaacttcaaatttttggacgattttccggcgattccgatgatcctgacttacacccgcatcccgcgtagcaccaaggatctggttgcacgcgttcgtgtactcgtgaccgaaaaattcccggaggttatgaaaccgatcctggatgcaatgggtgagtgcgcgctgcagggattagaaatcatgaccaaactgtcgaagtgtaaaggtacggacgacgaagctgttgaaacaaacaacgaactgtatgaacagctgctggaactgatccgtatcaaccacggcctgctggtcagcattggtgtgagccacccgggcctggaactgattaaaaatctttcggatgacctgcgcattggttctaccaagctgactggtgctggcggcggtggctgttctctgaccctcctgcgtcgcgatattacccaggaacaaatcgactcgttcaaaaaaaaactgcaggatgacttttcttatgaaaccttcgaaaccgacctgggcggtaccggctgttgtctcctgtccgccaaaaacttgaacaaagatctgaaaatcaaatctctcgtctttcagctgtttgaaaacaaaactaccaccaaacaacaaattgatgacctgctgctcccgggcaacaccaacttaccgtggacttcctaa

SEQ ID NO: 4 - DNA, 1356 bp, synthetic construct
  source 1..1356: mol_type = other DNA
atgtctgagcttcgcgctttctccgctccgggcaaggccctgctggccggaggctatctggtgctggacaccaaatatgaagcgtttgtagttggtctgtctgcccgtatgcatgcggtcgcgcacccgtatgggtcgctgcagggttcagataaattcgaggtgcgagtgaaaagcaaacagtttaaagacggtgagtggctgtatcacattagcccgaaatctggttttatcccggtatccatcggcggttccaaaaacccgttcattgaaaaagttattgccaacgttttctcctattttaaacctaacatggatgactactgtaaccgtaacctgttcgtgattgatattttctctgacgatgcttaccattcccaggaagacagcgttacggaacaccgtggcaaccgtcgtctgtcgtttcattcccaccgtatcgaagaagttccgaagactggcctgggtagctctgcaggcctggttaccgtcctgactactgctctggcctctttttttgtgtcggatctggaaaacaacgttgacaaatatcgtgaggtaattcataacctggctcaggtcgcacactgccaggcgcagggcaaaatcggctccggtttcgatgttgctgcggcagcttatggctccattcgttaccgtcgcttcccgcctgctctgatctcaaacctgccggatattggtagcgcaacctacggatcgaagctggctcacctggtggatgaggaagattggaatatcaccattaaatctaaccacctgccgtctggcctgaccctgtggatgggtgatatcaaaaacggctctgaaaccgtcaaactggtacagaaagttaagaattggtatgattctcacatgccggaatccctgaaaatctacaccgagctggatcacgcgaactcacgtttcatggacggtctgtccaaactggaccgtctgcacgaaacccacgatgattacagcgaccagatcttcgaatctctggaacgtaacgactgcacctgtcaaaaatacccggaaatcaccgaagttcgtgacgcggtagcgaccatccgccgctctttccgtaaaatcactaaggaaagcggcgctgacatcgaaccgccggttcagacctccctgctggacgattgccagactctgaaaggggtgctgacctgtctgattccgggtgcgggtggttatgacgctatcgcagtgatcacgaaacaggatgtagacctgcgtgcgcagactgcaaacgacaaacgttttagcaaagtacaatggctggatgttacccaggcggattggggtgttcgtaaagaaaaggaccctgaaacctacctggataaataa

SEQ ID NO: 5 - DNA, 1191 bp, synthetic construct
  source 1..1191: mol_type = other DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatcccgtatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcttagtcgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 6 - DNA, 573 bp, Escherichia coli
  source 1..573: mol_type = genomic DNA
atgcatcatcatcaccatcacgagctccaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 7 - DNA, 1509 bp, synthetic construct
  source 1..1509: mol_type = other DNA
atggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 8 - protein, 502 aa, Saccharomyces cerevisiae
MVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 9 - DNA, 30 bp, Agrocybe pediades
  source 1..30: mol_type = genomic DNA
aaagaacagctgcaggtatccatgaaggca

SEQ ID NO: 10 - DNA, 57 bp, Agrocybe pediades
  source 1..57: mol_type = genomic DNA
cgcccgctggacccggcgtacgtgaccaaagaacagctgcaggtatccatgaaggca

SEQ ID NO: 11 - protein, 296 aa, Agrocybe pediades
MFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 12 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYFLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 13 - protein, 85 aa, Abies grandis
AYSAMATMGYNGMAASCHTLHPTSPLKPFHGASTSLEAFNGEHMGLLRGYSKRKLSSYKNPASRSSNATVAQLLNPPQKGKKAVE

SEQ ID NO: 14 - DNA, 31 bp, synthetic construct
  source 1..31: mol_type = other DNA
taatacgactcactataggggaattgtgagc

SEQ ID NO: 15 - DNA, 43 bp, synthetic construct
  source 1..43: mol_type = other DNA
aaattaatacgactcactaatggggaattgtgagcggataaca

SEQ ID NO: 16 - DNA, 43 bp, synthetic construct
  source 1..43: mol_type = other DNA
aaattaatacgactcactcgaggggaattgtgagcggataaca

SEQ ID NO: 17 - DNA, 43 bp, synthetic construct
  source 1..43: mol_type = other DNA
aaattaatacgactcactataaaggaattgtgagcggataaca

SEQ ID NO: 18 - DNA, 43 bp, synthetic construct
  source 1..43: mol_type = other DNA
aaattaatacgactcactataggggaattgtgagcggataaca

SEQ ID NO: 19 - DNA, 43 bp, synthetic construct
  source 1..43: mol_type = other DNA
aaattaatacgactcactacagacgaattgtgagcggataaca

SEQ ID NO: 20 - DNA, 43 bp, synthetic construct
  source 1..43: mol_type = other DNA
aaattaatacgactcactagcggagaattgtgagcggataaca

SEQ ID NO: 21 - DNA, 43 bp, synthetic construct
  source 1..43: mol_type = other DNA
aaattaatacgactcactaacaccgaattgtgagcggataaca

SEQ ID NO: 22 - skipped

SEQ ID NO: 23 - DNA, 30 bp, synthetic construct
  source 1..30: mol_type = other DNA
tttgtttaactttaagaaggagatatacat

SEQ ID NO: 24 - DNA, 32 bp, synthetic construct
  source 1..32: mol_type = other DNA
gagatataataacgagataaggaaaagacaaa

SEQ ID NO: 25-26 - skipped (2 entries)

SEQ ID NO: 27 - protein, 335 aa, Lactarius deliciosus
PLAQWPWPRKLNQYYAEVKPESDQWIHGFGALDPKSQRSFDLCNFSLLGSLVYPLLDKDGVRVGCDLMVLFFIYDEFTDKVDGDGARVYAEMVMDAIRDPHKERPQGEPKLGEITRQFWLRAMKVSSAEAQRRFITTFAEYVYAVIDEASDRANGRVRGVEDYLKLTRLTAGGYPSFLAAEAGLNIPDEVMAHPALQTILSLAAESLVLTNDMYSYNIEQASGHGGHNIVTVIMNEKGVDLDGALNWLAEYHGQVLSNFQAQHRLLPSWGPEVDADVGAFVERLAYWIRGIDCWSLETERYFGTKGPEIKEHRRVTLLPKVKKPDVTPMMAQLNA

SEQ ID NO: 28 - protein, 344 aa, Psilocybe cyanescens
MSSTQFIIPDLLVNWPWLRVIDPNLQQVTDEANEWVESLDLFDPSQFKKFKGCDFNRLGALVGHLQGKEHLRISCDLMNFYFAFDEYTDLADKDEAMKISKDVMNTFKHTEVPFDNKLIEMARQFFKRTIDVVGEDNPGFERFIADFDAYTRSIIQEADDRDEGYIRSVDEYFILRRDTCGAKPSFSFHGLGLRIPNEVFEHPLVISMLEGATDLIAITNDMHSYGLEYSRGLDGHNVITAIMKEYEVDLQAALYWLSGYATKTISKFLTDRRKLPSWGPEIDVAVHEFFERVGRCVRGYDAWSYETNRYYGKNGLKIQETKRITLQPRDGAYITKERLQSSLA

SEQ ID NO: 29 - protein, 344 aa, Psilocybe cyanescens
MPRQYVIPDLLITWPWPRAINSSLTEVDEEANAWVQSLDLFDSAQFKKFKACNFNLLGALVGPLRSKEHLRISCDLMNFYFAFDEYTDLANREEAIKIAKDVMHAFRDTATPSDSKLIEMSRQFFRRTVDVVGDDKPGFERFIADFDAYTTSIIQEADDRAAGHIRNVEDYFILRRDTCGAKPSFSFYALGLNIPDDVFENPLVISMLEGATDLIAITNDMHSYGLEHSRGLDGHNVITAIMKEYNLDLQGALYWLSGYATKTIAKFVSDRKKLPSWSPSVDAGVREFFDLVGRCVRGYDAWSYETKRYYGDKGLKIQKTRRITLQPRDAAYITKEQLKVSIAA

SEQ ID NO: 30 - protein, 348 aa, Hypholoma fasciculare
MSQSQFTIPDLLASWPWPRAKNPALDQNLEDEANAWVASLELFEPRQLDKFKACQFNLLASLVGPIEGRDSLRISCDLMNFYFAFDEYTDVVSGDEVMMIVADVIQAFRDRESPEGSSKIKEMARQFFQRTIALVGEDTQGIDHFIADFEAYAKSVVQEADDRVQGIVRNVEEYFILRRDTCGGKPSFSFFGLGLCIPKEVFDHPVMQSLTESATDLIAMINDMHSYALEHARGLDGHNVITAIMHEHSVDLQGAFYWLSGHASKTVSKFLNDRKNLPSWGSDIDKAVNEYIDRMARCVRGYDAWSYETNRYYGKNGLEVQKSRKIMLQHRELEMGYITRDQLLIGAA